(19) United States
(12) Patent Application Publication
(10) Pub. No.: US 2026/0166036 A1
(43) Pub. Date: Jun. 18, 2026

(54) PROMOIETY STRATEGY TO ENHANCE DRUG ACTIVITY

(71) Applicant: University of Southern California, Los Angeles, CA (US)

(72) Inventor: Charles E. McKENNA, Los Angeles, CA (US)

(73) Assignee: University of Southern California, Los Angeles, CA (US)

(21) Appl. No.: 19/537,177

(22) Filed: Feb. 11, 2026

Related U.S. Application Data
(63) Continuation-in-part of application No. 18/021,753.
(60) Provisional application No. 63/067,204, filed on Aug. 18, 2020.

(51) Int. Cl. A61K 31/513; A61K 31/4709; A61K 47/60

(52) U.S. Cl. CPC A61K 31/513; A61K 31/4709; A61K 47/60

(57) ABSTRACT

Various nucleoside phosphate and phosphonate analogues are provided for treatment of viral infections. Methods of preparing the analogues, pharmaceutical compositions containing the analogues, and methods of using the analogues as antiviral compounds, especially against adenoviruses, coronaviruses, and varicella zoster viruses, are also provided.

### CROSS-REFERENCE TO RELATED APPLICATIONS

This application is a continuation-in-part of U.S. application Ser. No. 18/021,753 filed Feb. 16, 2023, which is the U.S. national phase of PCT Appln. No. PCT/US2021/046486 filed Aug. 18, 2021 which, in turn, claims the benefit of U.S. provisional application Ser. No. 63/067,204 filed Aug. 18, 2020, the disclosures of which are hereby incorporated in their entirety by reference herein.

### STATEMENT REGARDING FEDERALLY SPONSORED RESEARCH OR DEVELOPMENT

This invention was made with Government support under Contract/Grant Nos. HHSN272201100022I, HHSN272201000021I, HHSN27200007, HHSN272201100016I, R21AI130927, and R01AI135122 from NIH DMID Services. The Government has certain rights in this invention.

### TECHNICAL FIELD

In at least one aspect, the present invention is related to inhibition of nucleotide-binding enzymes such as nucleic acid polymerases and kinases to prevent or treat diseases.

### BACKGROUND

Nucleotide drugs are well-established in the prevention and treatment of diseases such as virus infections and cancer. A drawback of this approach is the poor bioavailability, low cell penetration, and toxicity of many nucleotide analogs, owing to their polarity and the charge of their ionized phosphate or phosphonate group(s). This can be exemplified by nucleoside phosphonate drugs used to treat virus infection.

Viral infection remains an evolving constellation of critical unmet health challenges. An example is human adenovirus (Ad) which has a linear duplex DNA genome that encodes about 35 genes and is enclosed in a protein capsid without a lipid membrane (1).

Adenovirus (Ad) is ubiquitous, infects most children, and generally causes asymptomatic or symptomatic infection of the respiratory, gastrointestinal, ocular, and other tissues that are usually self-limiting in healthy individuals with the exception of epidemic keratoconjunctivitis (1). However, Ad can cause serious infection in severely immunosuppressed individuals, especially in pediatric patients undergoing allogeneic hematopoietic stem cell transplantation (allo-HSCT), where the incidence of infection ranges from 5%-6% to 42%-47% depending on the study (2, 3). Mortality rates are up to 26% with symptomatic infection and up to 80% for disseminated disease (2, 3). In solid organ transplants, incidence ranges from 4% to 10% in pediatric liver transplants and up to 57% in small bowel recipients. Mortality can be as high as 18% in kidney transplants and 53% in liver transplants. With disseminated Ad disease, there is multi-organ involvement with Ad detected in peripheral blood, urine, bronchoalveolar fluid, and cerebrospinal fluid; death is associated with multi-organ failure and persisting or increasing levels of Ad in peripheral blood. Risk factors for Ad disease and death include young age, receipt of a mismatched and/or T cell-depleted graft, early and persistent isolation of Ad from multiple sites, high level of Ad in the blood, and increasing levels of Ad in stool beyond 106 genome copies per gram (2).

Despite the seriousness of Ad disease, there are no drugs currently approved to treat Ad infections. Intravenous gamma globulins have been employed, and intravenous cidofovir (CDV, HPMPC, (S)-1-(3-hydroxy-2-phosphonomethoxypropyl) cytosine) is used in many transplant clinics (4, 5), but controlled studies on the efficacy of these treatments have not been conducted. The DNA viruses are comprised of at least six distinct families of highly diverse viruses. The DNA polymerases that direct the replication of their genomes are well conserved and it is this characteristic that transcends the biologic differences among these viruses (6). This enzyme renders them susceptible to nucleoside phosphonate (NP) analogues that represent the predominant class of inhibitors for the treatment of DNA virus infections. CDV, a nucleoside phosphonate analogue of cytosine monophosphate (FIG. 1) is converted in cells to the CDV diphosphate analogue of dCTP, which is a preferred substrate for the Ad DNA polymerases, leading to chain termination and blocking viral replication.

Although CDV could in principle protect the target immunocompromised population from clinical manifestations associated with the DNA viruses, its clinical utility is limited by its modest efficacy, very low oral bioavailability (<5%) mandating i.v. or i.p. administration, and its dose limiting toxicity (7). CDV exhibits poor cellular uptake and is a substrate for organic anion transporter 1, leading to accumulation of CDV in renal tubules which causes nephrotoxicity (8).

A simple C16 ether-linked propyl ester prodrug of CDV, brincidofovir (FIG. 1) (HDP-CDV, CMX001, BCV), has displayed improved efficacy against all the DNA viruses tested (9). BCV does not accumulate in renal tubules and shows little or no nephrotoxicity (10) but GI irritation has been a problematic serious side effect, suggesting premature activation of a portion of the oral dose in the GI tract (11, 12). BCV inhibits the replication of multiple Ad types in cell culture, with EC50 values near 0.02 μM (13). BCV (and CDV) was also effective against Ad5 replication and pathogenesis in an immunosuppressed Syrian hamster model (14, 15). While BCV successfully suppressed infections during the first 10 weeks of administration, in a recent phase II clinical trial, infections arose after therapy was discontinued. BCV has also been recently evaluated in a randomized placebo-controlled phase II clinical trial as a preemptive treatment for pediatric and adult allogeneic hematopoietic stem cell transplant (allo-HSCT) patients and patients with asymptomatic Ad viremia (16, 17). BCV treatment reduced Ad viremia in some of these patients; however, the result was not statistically significant (17) and symptomatic GI irritation was observed. Drug-induced diarrhea was also observed as a serious side effect of BCV in a phase 3 trial with CMV-infected patients (18, 19). As a result, the trials were discontinued (11).

Another example is SARS-COV-2 which is responsible for a respiratory infection that can progress to severe pneumonia. COVID-19 has an estimated mortality rate of approximately 2-3.5%, which increases with age and the presence of comorbidities (e.g., hypertension, cardiac insufficiency, diabetes, and asthma). (20) Currently, laboratories and medical teams worldwide have focused on the repurposing of Food and Drug Administration (FDA)-approved drugs to treat the most severe cases of COVID-19, since there are no specific chemotherapeutic agents to treat this infection. Indeed, drug repositioning might be a short-term alternative to fight this disease. Since the efficacy, safety, and toxicity of these drugs are already well known, the initial phases of clinical trials could be skipped, which would reduce the cost and duration of the process. In general, drug repurposing is a cheaper, faster, and accessible way to make drugs available to the clinic. (21)

Several therapeutic agents have been evaluated for the treatment of SARS-Coronavirus-2 (SARS-COV-2) disease (COVID-19), but none have yet been shown to be efficacious. The FDA recently issued an emergency use authorization (EUA) for remdesivir for COVID-19 even as it awaits more concrete evidence of benefit. (22) A double-blind, randomized, placebo-controlled trial of intravenous remdesivir (200 mg loading dose on day 1, followed by 100 mg daily for up to 9 additional days or placebo for up to 10 days) in adults hospitalized with Covid-19 with evidence of lower respiratory tract infection was conducted. (23) Preliminary clinical trial results suggest that there may be a favorable benefit-risk profile for remdesivir compared with placebo in severe COVID-19 infection and further data on benefits would strengthen this evaluation. (24) However, given high mortality despite the use of remdesivir, it is clear that treatment with this antiviral drug alone is not likely to be sufficient. (23) Besides, remdesivir can be administered only to hospitalized patients with severe illness defined as patients with low oxygen in blood or needing breathing assistance.

### SUMMARY

The class of nucleotide analogue drugs includes both phosphonates and phosphates and is useful for the treatment of viral infections and cancer. Generally, these drugs suffer from lack of oral bioavailability and other drawbacks arising from high polarity. Viral infections have been of increasing concern in a public health context due to the emergence of new pathogens and the potential for development of drug resistance among currently recognized viruses. Thus, the development of new, more effective antiviral compounds effective against existing and emerging pathogens is a high priority in medical research. Viral replication depends on the function of a viral DNA or RNA polymerase. The DNA viruses, which include herpes, cytomegalo-, varicella zoster, adeno-, pox, polyoma and papilloma viruses, have a requirement for DNA synthesis during their life cycle. This common element therefore becomes a target for broad-spectrum antiviral compounds. Certain nucleoside phosphonates (NP), exemplified herein by cidofovir and its adenine analogue, HPMPA, have shown strong activity across a wide spectrum of DNA viruses. However, their development as antiviral drugs has been hampered by their inherent lack of bioavailability due to their highly polar nature. Our research at the University of Southern California (USC) has centered on overcoming the lack of oral bioavailability, low cellular permeability and tissue-specific toxicities of nucleoside phosphonate (NP) drugs by means of a novel prodrug strategy. These efforts have yielded a series of prodrugs of CDV and HPMPA with greatly enhanced in vitro antiviral potency against several DNA viruses, good oral absorption and significantly reduced risk of toxicity. Herein we disclose novel structural modifications of these prodrugs allowing their efficacy and pharmacological properties to be tuned advantageously. The invention of these useful promoiety modifications provides a wide range of compounds containing a modified phosphate, phosphonate or phosphinate group, including cyclic and acyclic NPs, with enhanced potency, oral availability, metabolism, cellular permeability and solubility for inhibiting pathogenic viruses. Furthermore, it can be applied to enhance the bioavailability and potency of nucleotide analogues designed to block repair polymerases in cancer cells to overcome their resistance to DNA-damaging chemotherapeutic drugs.

The RNA-dependent RNA polymerase (RdRp) of coronaviruses is a well-established drug target; the active site of the RdRp is highly conserved among positive-sense RNA viruses. These RdRps have low fidelity allowing them to recognize a variety of modified nucleotide analogues as substrates. Such nucleoside triphosphate analogues may inhibit further RNA polymerase catalyzed RNA replication making them important candidate anti-viral agents. Recent studies examined a library of nucleoside triphosphate (phosphonate) analogues for incorporation by the RdRps of SARS-COV-2. (25) Of the 11 tested, 6 exhibited complete termination of the polymerase reaction, 2 showed incomplete or delayed termination. Five among them are triphosphate analogues of nucleoside drugs that are FDA approved medications for treatment of viral infections with established toxicity profiles. Thus, these five drugs and the parent nucleoside phosphate of remdesivir offer a molecular basis for the synthesis of prodrugs and their evaluation in SARS-COV-2 virus inhibition. It will be appreciated by those skilled in the art that this list is not limiting, in that the prodrug approach disclosed in this invention can be readily used with any nucleoside phosphonate or nucleoside phosphate drug or drug precursor molecule.

During DNA/RNA replication, nucleosides are phosphorylated by various host cell kinases into their active triphosphate form, then are taken up by polymerases and incorporated into the growing chain. The same process is required for modified nucleosides, however, one of the major limitations to utilizing nucleosides as drugs is the specificity of the kinases involved in the various phosphorylation steps. These steps then become rate limiting for the overall conversion to the active triphosphate form. Since nucleoside analogues are not always recognized efficiently and thus may initially appear inactive, it became important to design analogues that could overcome this issue. In that regard, utilization of monophosphate analogues would be the best choice, however, the phosphate group renders the nucleotides extremely polar, therefore making it difficult for the compounds to cross the cellular membrane. This condition limits the therapeutic scope of these drugs. Moreover, after intravenous injection drugs of this class tend to accumulate in the kidney leading to renal toxicity. (8) Thus, there is a need for more effective, orally bioavailable forms of these drugs. Several prodrug approaches to improve oral absorption of antiviral nucleoside analogues by incorporating various phosphate or phosphonate anion masking groups have been developed. (26)

Recently, certain “tunable” prodrugs of CDV synthesized at the Univ. of Southern California (USC) have been demonstrated to be several orders of magnitude more potent against four Ad serotypes in vitro and orally effective against Ad6 in a Syrian hamster model. (27)

N-alkylated tyrosinamide ester modifications of CDV and HPMPA were found to exhibit up to three logs higher potency against Ad in infected HFF cells than the parent drug (28). Two of these compounds (FIG. 2) were highly effective orally in an Ad6 infection animal model (28). In addition, one of these was shown to be effective in vivo against a different DNA virus, VZV (29).

In the present application, compounds that include prodrugs with differing side chains derived from a natural amino acid (tyrosine, serine, homoserine, threonine) in either L- or D-configuration are provided in the form of an amide in which the amide substituent side chain is an alkyl, alkyl ether, thioether, or alkene. Embodiments of the compounds have a range of effective lipophilicity values allowing variation in aqueous solubility, oral bioavailability, cell permeability and in vivo activation properties. The embodiments have promoieties derived from a single amino acid, which are expected to have low toxicity. The features described above also make possible a novel “precision medicine” approach to treatment of viral infections, whereby the prodrug variations can be exploited to match optimal activation of the prodrug to a given patient or strain of virus.

Achieving the right balance in a given drug of potency, lipophilicity and hydrophilicity, permeability, chemical stability, rate, loci and extent of activation, safety and key physical properties, such as solubility, is a challenge. The strategy disclosed here involves a non-toxic amino acid prodrug moiety offering exceptional versatility for “tuning” potency and pharmacological properties by appropriate modification at one or more of multiple functionalizable sites.

Embodiments of the invention include incorporation of an O-Ser, O-homo-Ser, O-Thr or O-Tyr amino acid phosphonate ester linkage to the parent drug. In some embodiments, the amino acid promoiety has the L-configuration (natural configuration). In other embodiments, the amino acid promoiety has the D-configuration, which can provide enhanced antiviral potency compared to the corresponding L-isomer, with further modification of said linking group with a lipophilic group.

In another aspect, a prodrug including a compound having formula (1) is provided:

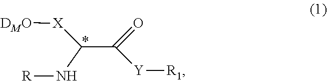

or a pharmaceutically acceptable salt thereof,
   wherein:
D_{M} is a drug moiety;
X is selected from the group consisting of C₁-C₄ alkylene, C₆-C₁₀ arylene, and C₇-C₁₂ alkylarylene (each of these are divalent);
Y is O or NH;
R is H, C₁-C₆ alkyl, or C(O)R₂ or a lipid-like group;
R₁ is H, C₁-C₂₅ alkyl, C₂-C₂₅ alkenyl, C₂-C₂₅ alkoxyalkyl, or C₂-C₂₅ alkylthioalkyl; and
R₂ is lipid-like.

In a refinement, the carbon labeled with * can provide an amino acid or amino acid derivative in the D form or the L form.

In another aspect, a nucleoside phosphonate (1) or nucleoside phosphate (2) is provided, having the formula

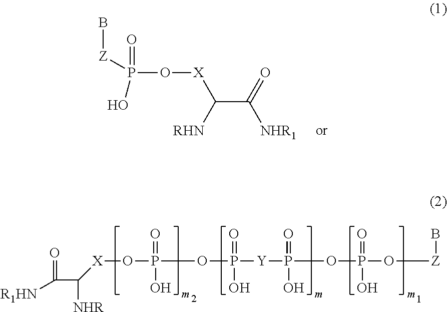

where: B is a natural or unnatural purine or pyrimidine base; X is CH₂Ph, CH₂, CH₂CH₂, or CHCH₃; Y is O, NH, CH₂, CHF, CHCl, CHBr, CF₂, CCl₂, CBr₂, CCH₃, C(CH₃)₂, CHN₃, CCH₃N₃; Z is a cyclic or acyclic sugar, such as ribose or deoxyribose, or a related structure; m=0-1; m₁=0-3; m₂=0-3; R is H or C(O)R₂, wherein R₂ is lipid-like; and R₁ is H or lipid-like. In addition, the NHR₁ group in (1) may be replaced by OR₃, wherein R₃=a C₁-C₄ alkyl, when R═C(O)R₂, with R₂ defined as above.

In some embodiments: a) R₁/R₂ is alkyl, which can be a long-chain alkyl, which can be C₈₋₁₈ alkyl; b) R₁/R₂ is alkyl, alkene, ether or thioether, which can be C₁₂₋₁₈ alkyl, (CH₂)ₙCH═CHCₙ₁, (CH₂)ₙ—O—(CH₂)ₙ₁ or (CH₂)ₙ—S—(CH₂)ₙ₁ wherein n and/or n1=4-9; or any combination of a)-b).

In some embodiments, nucleoside phosphonates (3) and (4) are provided, having the formula

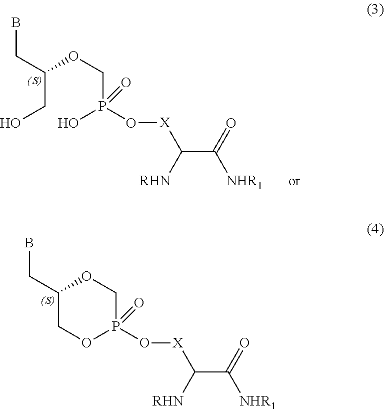

wherein B, X, R and R₁ are as defined in the two preceding paragraphs.

In some embodiments, B is an adenine or cytosine. In other embodiments B is a natural or unnatural purine or pyrimidine. B may form a natural or unnatural nucleoside.

In some embodiments of the foregoing, B and Z correspond to a nucleoside compound effective against virus infections or cancer. Examples of viruses are SARS-COV-2, adenovirus, cytomegalovirus and varicella zoster virus.

In some embodiments in formulas 3 and 4, R═H and R₁ is defined above.

In some embodiments in formulas 3 and 4, R₁═H or OR₃ and R═C(O)R₂ where R₃ and R₂ are defined as above.

In another aspect, a pharmaceutical composition that includes a nucleoside phosphonate is provided. The pharmaceutical composition includes one or more of the nucleoside phosphonates described herein, and a pharmaceutically acceptable carrier.

In a further aspect, a method of treating an adenovirus, coronavirus, varicella zoster or other virus infection in a subject in need thereof is provided. The method includes administering to the subject an effective amount of one or more of the nucleoside phosphonates described herein, or an effective amount of a pharmaceutical composition containing one or more of the nucleoside phosphonates described herein. The subject can be a human or other animal, such as another mammal.

In some embodiments, a method of treating cancer is provided. The method includes administering to the subject an effective amount of at least one nucleoside phosphonate or nucleoside phosphate described herein.

The following Examples describe the synthesis of embodiments of the prodrug compounds. The present invention may be better understood by referring to the accompanying examples, which are intended for illustration purposes only and should not in any sense be construed as limiting the scope of the invention.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1 is a panel showing the structures of CDV, HPMPA and BCV.

FIG. 2 is a panel showing the structures of USC-505 and USC-087.

FIG. 3 is a synthetic scheme for making embodiments of the present disclosure.

FIG. 4 is a panel showing prodrug structures of GS-441524, ganciclovir, cidofovir, abacavir, stavudine, and entecavir with variable substituents X, Y, and Z.

FIGS. 5A, 5B, 5C, and 5D. Inhibition by USC-093, USC-093D of HAdV-C5 and HAdV-C6 in cell culture. A) Evaluation of USC-093, USC-093D, and CDV (positive control) against HAdV-C5 in HFF cells. B) Evaluation of USC-093 and USC-093D against HAdV-C5 in A549 cells. C) Evaluation of USC-093, USC-093D, and CDV against HAdV-C6 in HFF cells. D) Evaluation of USC-093 and USC-093D against HAdV-C6 in A549 cells.

FIGS. 6A, 6B, and 6C. A) Neither USC-093 nor USC-093D mitigated HAdV-C6-induced morbidity. Each symbol represents the group mean; the error bars depict the standard deviation. HAdV-C6+Vehicle vs. HAdV-C6+VGCV p<0.0001. B) USC-093D marginally mitigates the lung pathology induced by intranasal infection with HAdV-C6. Weight of the left lung lobe is shown. C) USC-093 marginally inhibits the replication of HAdV-C6 in the lung of immunosuppressed hamsters. The symbols represent data from individual animals, and the bar symbolizes the median. *: p<0.05

FIG. 7. Chemical structures of Cidofovir (CDV, HPMPC) and USC-150, an L-homoserinamide prodrug of HPMPC.

FIG. 8. Synthesis scheme of USC-150. The synthesis proceeds in four steps: (1) reaction of Boc-protected homoserine lactone with hexadecylamine in THF (83% yield); (2) coupling with CDV using PyBOP/DIEA in DMF (74% yield); (3) Boc deprotection with HCl/THF (84% yield); (4) ring opening with aqueous NH4OH/MeCN (57% yield).

FIGS. 9A and 9B. Delayed treatment with USC-150 is efficacious against HAdV-C6 infections in hamsters. A. Survival. HAdV-C6+Vehicle vs. all USC-150-treated groups p<0.0001 (log rank test) B. Body weight changes. The symbols represent the group mean; the error bars signify the standard deviation. No group means were calculated for a given group once an animal was sacrificed moribund. **: p<0.01

FIGS. 10A and 10B. USC-150 inhibits the replication of HAdV-C6 in the liver of hamsters and mitigates adenoviral pathogenesis even when the administration of the drug started at 4 days post challenge. A. Serum alanine transaminase levels. ***: p<0.001 (two-tailed Mann-Whitney test) B. Virus burden in the liver. For both graphs, symbols represent data from individual animals; the horizontal bar represents the mean. Samples that were collected one day before the scheduled sacrifice time are also shown.

FIG. 11. Structures of USC-374 and its parent nucleotide analogue HPMPA.

FIG. 12. Gram-scale synthesis scheme of USC-374.

FIG. 13. Low-Dose USC-374 s.c. Mitigates HAdV-C6-Induced Weight Loss. Weight changes in immunosuppressed hamsters infected with HAdV-C6, showing how USC-374 (10 mg/kg q.d.) mitigates infection-induced morbidity. Each symbol represents the group mean; error bars denote standard deviation. Statistically significant differences between HAdV-C6+Vehicle and HAdV-C6+USC-374 s.c. (p<0.001, two-way ANOVA) demonstrate the prophylactic benefit of USC-374 in preserving body weight.

FIG. 14. Low-Dose USC-374 (s.c.) Alleviates HAdV-C6-Induced Lung Pathology: Lung Weight as a Surrogate Measure. USC-374 (10 mg/kg) mitigates the lung pathology induced by intranasal HAdV-C6 infection in immunosuppressed hamsters. Symbols represent individual animals; horizontal bars denote the median. Lung weights were significantly lower in the HAdV-C6+USC-374 s.c. group compared to HAdV-C6+Vehicle (p<0.05, Mann-Whitney U-test).

FIG. 15. TCID50 Assay: Low-Dose USC-374 (s.c.) Suppresses HAdV-C6 Lung Replication. Lung viral burden in intranasally infected hamsters measured by TCID50. Symbols represent individual animals, with horizontal bars indicating the median. USC-374 (10 mg/kg, s.c.) lowered HAdV-C6 replication relative to the vehicle control, supporting its efficacy at a lower dose than the standard comparator.

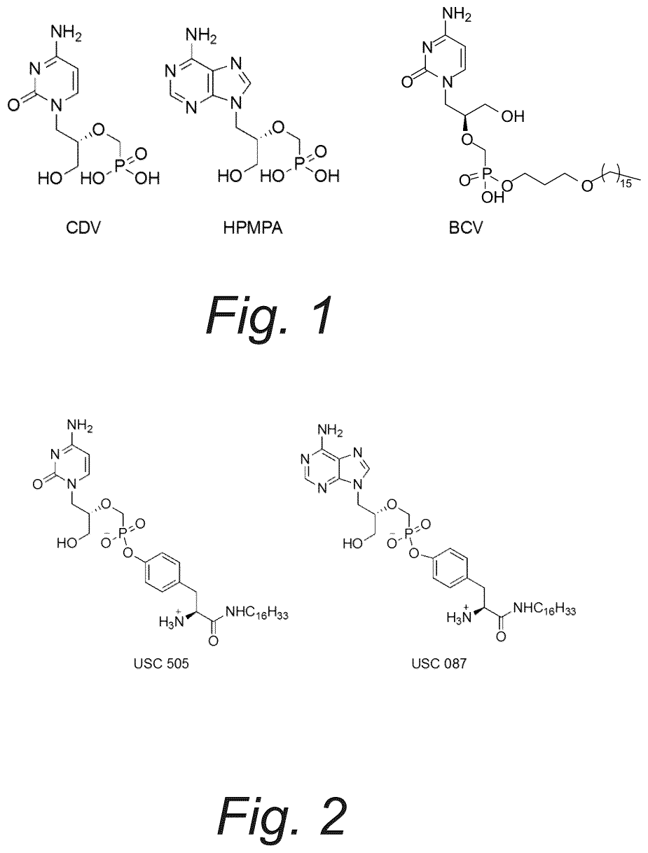

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Ri where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups; single letters (e.g., “n” or “o”) are 1, 2, 3, 4, or 5; in the compounds disclosed herein a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

For all compounds expressed as an empirical chemical formula with a plurality of letters and numeric subscripts (e.g., CH₂O), values of the subscripts can be plus or minus 50 percent of the values indicated rounded to or truncated to two significant figures. For example, if CH₂O is indicated, a compound of formula C_{(0.8-1.2)} H_{(1.6-2.4)} O_{(0.8-1.2)} is also contemplated. In a refinement, values of the subscripts can be plus or minus 30 percent of the values indicated rounded to or truncated to two significant figures. In still another refinement, values of the subscripts can be plus or minus 20 percent of the values indicated rounded to or truncated to two significant figures.

The compounds set forth herein may be used per se or as pharmaceutically acceptable derivatives. The latter term includes salts, esters, and other derivatives generally considered acceptable by pharmaceutical standards. Useful derivatives, for example, include salts of organic and inorganic acids such as sulfates, phosphates, hydrohalide salts, carboxylate salts, etc., as well as esters of carboxylic acid or hydroxyl substituents, ethers of hydroxyl substituents, amides of amino substituents, as well as carbamates, ureas, etc. Synthesis of these derivatives is conventional, and well known to those skilled in pharmaceutical chemistry. For example, compounds bearing hydroxyl groups may be converted to esters by customary techniques of organic chemistry, such as reaction with an acyl halide, carboxylic acid anhydride, or by esterification with an acid while removing byproduct water. In some cases, derivation may be desired to facilitate compounding of the pharmaceutical into an acceptable form such as tablets, powder, aqueous dispersion, capsule, etc., or may be useful in assisting bioavailability of the drug following administration, for example, by rendering the compound more or less soluble. In many cases, such as, for example, esters, ureas, carbamates, ethers, etc., the derivative may act as “prodrug,” which liberates the active form by biological transformation, i.e., by enzymatic hydrolysis of an ester functionality, as is well known to the pharmaceutical chemist.

Typical dosages for mammalian species may vary from 0.001 mg/Kg of body weight to about 100 mg/Kg of body weight, preferably 0.01 mg/Kg to 5 mg/Kg. The actual amount will vary depending upon the particular therapeutic activity desired to be altered, and the desired degree of alteration. The upper limits may, as with virtually all drugs, be limited by toxicity of the drug or its metabolites, or by the presence of unwanted side effects. The drugs may be administered in any form, but preferably in the form of tablets or capsules with appropriate excipients. Dosages, forms of administration, etc., can be readily determined by those skilled in the art.

All groups recited herein that include a range of carbon atoms are intended to encompass each and every possible sub-range therein. For example, C₁-C₂₅ alkyl includes C₁-C₅ alkyl, C₃-C₂₀ alkyl, C₄-C₂₅ alkyl, C₅-C₂₅ alkyl, C₁-C₁₅ alkyl, and the like.”

Throughout this application, where patents, applications, and publications are referenced, the disclosures of these publications in their entireties are hereby incorporated by reference into this application to more fully describe the state of the art to which this invention pertains.

As used herein, the term “derivative” when used in reference to a purine base or pyrimidine base refers to a modified heterocyclic nucleobase that retains either: (i) the core bicyclic purine ring system including fused pyrimidine and imidazole rings, or (ii) the core monocyclic pyrimidine ring system. A derivative of a purine or pyrimidine base includes one or more of the following modifications relative to adenine, guanine, cytosine, thymine, or uracil:
(a) substitution at any ring carbon or exocyclic position with a substituent selected from halogen (F, Cl, Br, I), amino, alkylamino, dialkylamino, hydroxyl, alkoxy, thiol, alkylthio, cyano, nitro, azido, carboxamido, alkyl, haloalkyl, or aryl;
(b) replacement of a ring carbon with nitrogen (aza modification), including but not limited to 5-azacytosine, 6-azauracil, 6-azathymine, 8-azaadenine, and 8-azaguanine;
(c) replacement of a ring nitrogen with carbon (deaza modification), including but not limited to 7-deazaadenine, 7-deazaguanine, 3-deazaadenine, and 3-deazaguanine;
(d) replacement of a ring carbonyl oxygen with sulfur (thio modification), including but not limited to 2-thiouracil, 4-thiouracil, 2-thiothymine, 6-thioguanine, and 6-thiopurine;
(e) replacement of an exocyclic amino group with a hydroxyl group or vice versa, including but not limited to hypoxanthine (inosine base), xanthine, and 2-aminopurine;
(f) addition of substituents at the 5-position of pyrimidines, including but not limited to 5-fluorouracil, 5-fluorocytosine, 5-bromouracil, 5-iodouracil, 5-methylcytosine, 5-hydroxymethylcytosine, and 5-propynyl-uracil;
(g) addition of substituents at the 8-position of purines;
(h) fusion of an additional carbocyclic or heterocyclic ring to the nucleobase core, including but not limited to benzimidazole, benzotriazole, and tricyclic nucleobase analogues;
(i) saturation or partial saturation of one or more ring bonds; or
(j) combinations of two or more of modifications (a) through (i).
   Such derivatives retain the capacity to participate in hydrogen bonding interactions characteristic of nucleobases when incorporated into nucleosides or nucleotides and, in the context of the prodrugs disclosed herein, provide substrates that are recognized by viral or cellular polymerases, kinases, or other nucleotide-processing enzymes. The term “derivative” as applied to nucleobases does not require that the modified base exhibit Watson-Crick base pairing, as certain therapeutically useful nucleobase analogues function through mechanisms other than incorporation into nucleic acids, such as chain termination or polymerase inhibition.

As used herein, the term “lipid-like” refers to a substituent that imparts lipophilic character to the molecule, thereby enhancing membrane permeability and cellular uptake. A lipid-like substituent is a hydrocarbyl group or modified hydrocarbyl group having at least 8 carbon atoms and including: (a) a straight-chain or branched alkyl group; (b) a straight-chain or branched alkenyl group containing one or more carbon-carbon double bonds; (c) an alkoxyalkyl group (alkyl-O-alkyl); (d) an alkylthioalkyl group (alkyl-S-alkyl); or (e) combinations thereof. In some embodiments, a lipid-like substituent has from 6 to 24 carbon atoms, or from 12 to 20 carbon atoms, or from 14 to 18 carbon atoms. The term encompasses saturated and unsaturated groups, and groups wherein one or more methylene units are replaced by an oxygen or sulfur atom, provided that the overall chain length and hydrophobic character are sufficient to promote partitioning into lipid bilayers. For purposes of this definition, “hydrocarbyl” refers to a monovalent radical containing only carbon and hydrogen atoms, and “modified hydrocarbyl” refers to a hydrocarbyl group in which one or more carbon atoms have been replaced by a heteroatom selected from oxygen and sulfur. In some refinements, the term “lipid-like” includes C₁₀-C₂₆ aryl groups and C₆-C₂₆ alkyl groups, wherein the alkyl groups optionally have 1 to 4 degrees of unsaturation.

As used herein, the term “drug moiety” (D_{M}) refers to a therapeutically active compound or pharmacophore that is covalently linked through an oxygen atom to the promoiety via linker X. The drug moiety includes any compound bearing a functional group capable of forming a covalent bond to oxygen, wherein said oxygen atom is bonded to linker X as shown in the prodrug structure. Drug moieties include, but are not limited to:
(a) Phosphorus-containing drug moieties, wherein a phosphate, phosphonate, or phosphinate group of the drug moiety is bonded to the oxygen atom, including:
(i) acyclic nucleoside phosphonates, such as cidofovir (CDV, (S)-HPMPC), (S)-HPMPA, adefovir, tenofovir, and analogues thereof;
(ii) cyclic nucleoside phosphonates, such as cyclic cidofovir (cCDV), cyclic(S)-HPMPA, and analogues thereof;
(iii) nucleoside monophosphates derived from natural or unnatural nucleosides, including monophosphates of remdesivir, sofosbuvir, gemcitabine, cytarabine, fludarabine, cladribine, clofarabine, and analogues thereof;
(iv) nucleoside diphosphates, triphosphates, and non-hydrolyzable analogues thereof wherein one or more bridging oxygen atoms are replaced by CH₂, CHF, CHCl, CHBr, CF₂, CCl₂, CBr₂, NH, or S;
(v) non-nucleoside phosphates and phosphonates, including fosfomycin, bisphosphonates, and phosphorylated small molecules;
(b) Carboxylic acid-containing drug moieties, wherein a carboxyl group of the drug moiety forms an ester bond to the oxygen atom, including:
   (i) non-steroidal anti-inflammatory drugs (NSAIDs) such as ibuprofen, naproxen, ketoprofen, flurbiprofen, and indomethacin;
   (ii) statins such as atorvastatin, pravastatin, rosuvastatin, and simvastatin acid;
   (iii) angiotensin-converting enzyme (ACE) inhibitors such as enalaprilat, lisinopril, and ramiprilat;
   (iv) antivirals such as zanamivir, peramivir, and baloxavir acid;
   (v) antibiotics such as β-lactams, fluoroquinolones, and cephalosporins bearing carboxylic acid groups;
   (vi) antineoplastic agents such as methotrexate, pemetrexed, and chlorambucil;
(c) Hydroxyl-containing drug moieties, wherein a hydroxyl group of the drug moiety forms an ether or carbonate linkage to the oxygen atom, including:
   (i) nucleoside analogues such as acyclovir, ganciclovir, penciclovir, ribavirin, and zidovudine;
   (ii) steroids and hormones such as prednisolone, dexamethasone, estradiol, and testosterone;
   (iii) taxanes such as paclitaxel and docetaxel;
   (iv) anthracyclines such as doxorubicin and daunorubicin;
   (v) cannabinoids and terpenoids bearing hydroxyl groups;
(d) Amine-containing drug moieties, wherein an amino group of the drug moiety forms a carbamate linkage to the oxygen atom (via —O—C(O)—NR—), including:
   (i) aminoglycoside antibiotics;
   (ii) kinase inhibitors bearing primary or secondary amino groups;
   (iii) dopaminergic agents and CNS-active compounds;
(e) Sulfonic acid or sulfonamide-containing drug moieties, wherein a sulfur-containing acidic group forms a bond to the oxygen atom;
(f) Any other drug moiety bearing a functional group capable of covalent attachment to oxygen, either directly or through a linking group, wherein the resulting prodrug releases the active drug moiety upon enzymatic or chemical cleavage in vivo.

The drug moiety may be effective for treating viral infections, bacterial infections, fungal infections, parasitic infections, cancer, inflammatory conditions, cardiovascular diseases, metabolic disorders, neurological disorders, or other diseases or conditions amenable to prodrug therapy. In embodiments where the drug moiety contains a nucleobase, said nucleobase may be a natural or unnatural purine or pyrimidine, including adenine, guanine, cytosine, thymine, uracil, and modified variants thereof.

“Acyclic sugar surrogate” or “acyclic sugar mimic” refers to an acyclic polyhydroxylated carbon chain that replaces the cyclic ribose or deoxyribose moiety typically found in natural nucleosides. Such surrogates are not carbohydrates but provide hydroxyl groups capable of phosphorylation and maintain spatial orientation sufficient for enzymatic recognition and biological activity. Alternatively, such groups may be referred to as an “acyclic linker moiety,” meaning an acyclic carbon chain bearing one or more hydroxyl groups that connects the nucleobase to the remainder of the molecule. The hydroxyl groups are positioned to permit phosphorylation by cellular or viral kinases.

“Cyclic phosphonate prodrug” or “cyclic phosphonate ester” refers to a prodrug moiety in which a phosphonate group forms a cyclic structure with one or more hydroxyl groups of the acyclic sugar surrogate. In one embodiment, a single hydroxyl group of the acyclic linker moiety participates in the cyclic phosphonate, leaving a second hydroxyl group free, thereby forming a six-membered ring containing phosphorus. In another embodiment, both hydroxyl groups of the acyclic linker moiety are engaged with the phosphonate, forming a bicyclic structure in which the phosphorus atom bridges both oxygen atoms. Such cyclic phosphonate prodrugs enhance cell membrane permeability and metabolic stability relative to the corresponding free phosphonate. Upon cellular uptake, the cyclic phosphonate is hydrolyzed by intracellular enzymes to release the active phosphonate metabolite.

“Cyclic sugar mimic” or “cyclic sugar surrogate” refers to a carbocyclic or heterocyclic ring system that replaces the cyclic ribose or deoxyribose moiety typically found in natural nucleosides but differs structurally from a natural furanose sugar. Such mimics include, but are not limited to, carbocyclic rings (wherein the ring oxygen of ribose is replaced by a methylene group), morpholine rings, oxetane rings, and other saturated or unsaturated ring systems bearing one or more hydroxyl groups or hydroxyl group equivalents capable of phosphorylation. Cyclic sugar mimics are not carbohydrates but maintain sufficient structural and spatial similarity to natural sugars to permit enzymatic recognition and biological activity.

In methods of treating virus infection or inhibiting virus replication, an effective amount, which can be a therapeutically effective amount, of an acyclic nucleoside phosphonate, or a salt or pharmaceutically acceptable salt thereof, may be administered. A therapeutically effective amount of a compound is an amount that results in an improvement or a desired change in condition for which a compound is administered, when the compound is administered once or over a period of time. For example, with respect to virus infections, the improvement can be a lowering of virus titer, or a reduction in the symptoms or discomfort associated with a viral infection. As is known, the amount will vary depending on such particulars as the type of virus infection, the condition being treated, the specific acyclic nucleoside phosphonate compound utilized, the severity of the condition, and the characteristics of the subject. The subject can be a person or another animal, such as another mammal.

An antiviral compound such as a nucleoside phosphonate can be prepared as a salt, which may be a pharmaceutically acceptable salt. Pharmaceutically acceptable salts are well known in the art and include salts prepared from pharmaceutically acceptable non-toxic acids, including inorganic acids and organic acids. Suitable non-toxic acids include inorganic and organic acids such as acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic acids, and the like. Salts formed with, for example, a POH group, can be derived from inorganic bases including, but not limited to, sodium, potassium, ammonium, calcium or ferric hydroxides, and organic bases including, but not limited to, isopropylamine, trimethylamine, histidine, and procaine.

Pharmaceutical compositions containing nucleoside phosphonates will typically contain a pharmaceutically acceptable carrier. Although oral administration is a desired route of administration, other means of administration such as nasal, topical (for example, administration to the skin or eye) or rectal administration, or by injection or inhalation, are also contemplated. Depending on the intended mode of administration, the pharmaceutical compositions may be in the form of solid, semi-solid or liquid dosage forms, such as, for example, tablets, suppositories, pills, capsules, powders, liquids, suspensions, drops, ointments, creams or lotions, preferably in unit dosage form suitable for single administration of a precise dosage. The compositions may include an effective amount of a selected compound in combination with a pharmaceutically acceptable carrier and, in addition, may include other pharmaceutical agents such as other anti-viral agents, adjuvants, diluents, buffers, and the like. The compound may thus be administered in dosage formulations containing conventional non-toxic pharmaceutically acceptable carriers, adjuvants and vehicles. The amount of active compound administered will be dependent on the subject being treated, the subject's weight, the manner of administration and the judgment of the prescribing physician.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions may, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan mono-laurate, triethanolamine acetate, triethanolamine oleate, etc. Actual methods of preparing such dosage forms are known, or will be apparent, to those skilled in this art. For oral administration, the composition will generally take the form of a tablet or capsule, or may be an aqueous or nonaqueous solution, suspension or syrup. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

In an embodiment, a prodrug including a compound having formula (1) is provided:

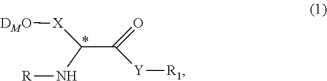

or a pharmaceutically acceptable salt thereof,
   wherein:
D_{M} is a drug moiety;
X is selected from the group consisting of C₁-C₄ alkylene, C₆-C₁₀ arylene, and C₇-C₁₂ alkylarylene (each of these are divalent);
Y is O or NH;
R is H, C₁-C₆ alkyl, or C(O)R₂ or a lipid-like group;
R₁ is H, C₁-C₂₅ alkyl, C₂-C₂₅ alkenyl, C₂-C₂₅ alkoxyalkyl, or C₂-C₂₅ alkylthioalkyl; and
R₂ is lipid-like. In a refinement, the carbon labeled with * can provide an amino acid or amino acid derivative in the D form or the L form. It should be appreciated that any substituent or selection for a group (e.g., D_{M}, X, R, R₁, R₂) in a specific compound can be imputed onto any compound described by formula (1).

In another aspect, R, R₁, and R₂ are each independently C₅ to C₂₅ alkyl, C₅ to C₂₅ alkylene, C₁₀ to C₂₆ alkylaryl, or C₁₀ to C₂₆ aryl. In a refinement, R, R₁, and R₂ are each independently C₈ to C₂₄ alkyl, C₈ to C₂₄ alkylene, C₁₀ to C₂₆ alkylaryl, or C₁₀ to C₂₆ aryl.

In another aspect, X is O and Ri is H or C₁-C₄ alkyl. In a further refinement, X is NH and R₁ is H or C₁-C₄ alkyl.

In another aspect, X is CH₂ (C₆H₄), CH₂, CH₂CH₂, or CHCH₃.

In another aspect, D_{M} is

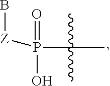

wherein B is a purine base, pyrimidine base, or derivative thereof and Z is a divalent moiety derived from a sugar, a cyclic sugar mimic or acyclic sugar mimic. In a refinement, Z is a monohydroxylated hydrocarbon chain (e.g., C₃-C₇ alkyl), polyhydroxylated hydrocarbon chain (e.g., C₃-C₇ alkyl), a monohydroxylated hydrocarbon ring (e.g., C₅-C₇ cycloalkyl), polyhydroxylated hydrocarbon ring. In a further refinement, Z includes 1, 2, 3, 4, or 5 hydroxyl groups whether Z is acyclic or cyclic.

In another aspect, Z is

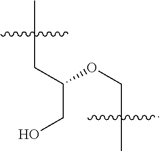 [SMILES: *CO[C@@H](C*)CO]

In another aspect, D_{M} is

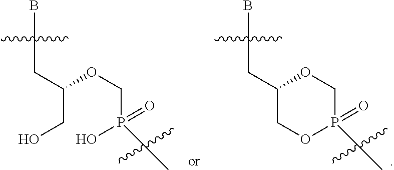

In another aspect, D_{M}O is:

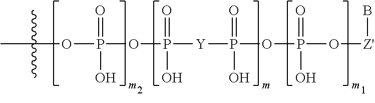

wherein
B is a purine base, pyrimidine base, or derivative thereof;
Z′ is a sugar, a cyclic sugar mimic or acyclic sugar mimic;
Y is O, NH, CH₂, CHF, CHCl, CHBr, CF₂, CCl₂, CBr₂, CCH₃, C(CH₃)₂, CHN₃, CCH₃N₃
m is 0 to 1 (e.g., 0 or 1);
m₁ is 0 to 3 (e.g., 0, 1, 2, or 3); and
m₂ is 0 to 3 (e.g., 0, 1, 2, or 3).

In another aspect, Z′ is a divalent moiety derived from a sugar, a cyclic sugar mimic or acyclic sugar mimic. In a refinement, Z′ is a monohydroxylated hydrocarbon chain (e.g., C₃-C₇ alkyl), polyhydroxylated hydrocarbon chain (e.g., C₃-C₇ alkyl), a monohydroxylated hydrocarbon ring (e.g., C₅-C₇ cycloalkyl), polyhydroxylated hydrocarbon ring. In a further refinement, Z′ includes 1, 2, 3, 4, or 5 hydroxyl groups whether Z′ is acyclic or cyclic. A specific example for Z′ is

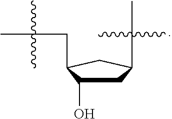

In another aspect, R is:

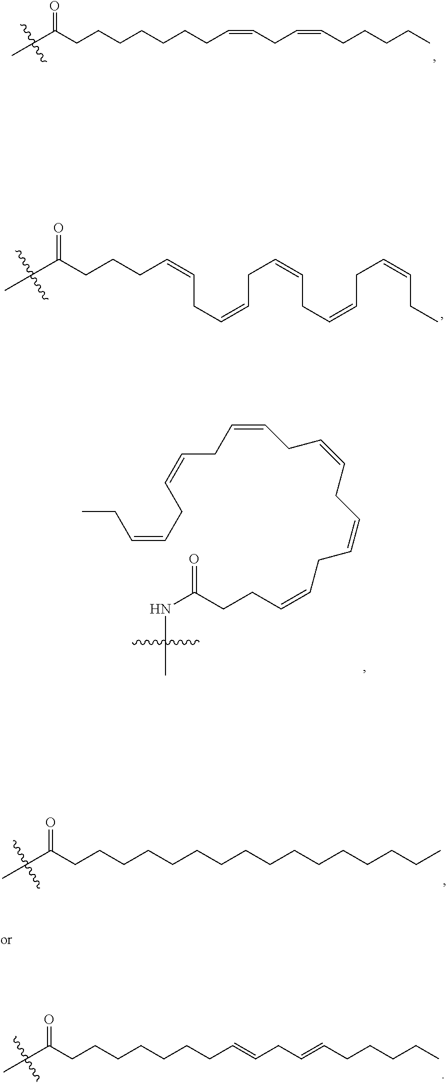

Specific examples of useful compounds include the following:

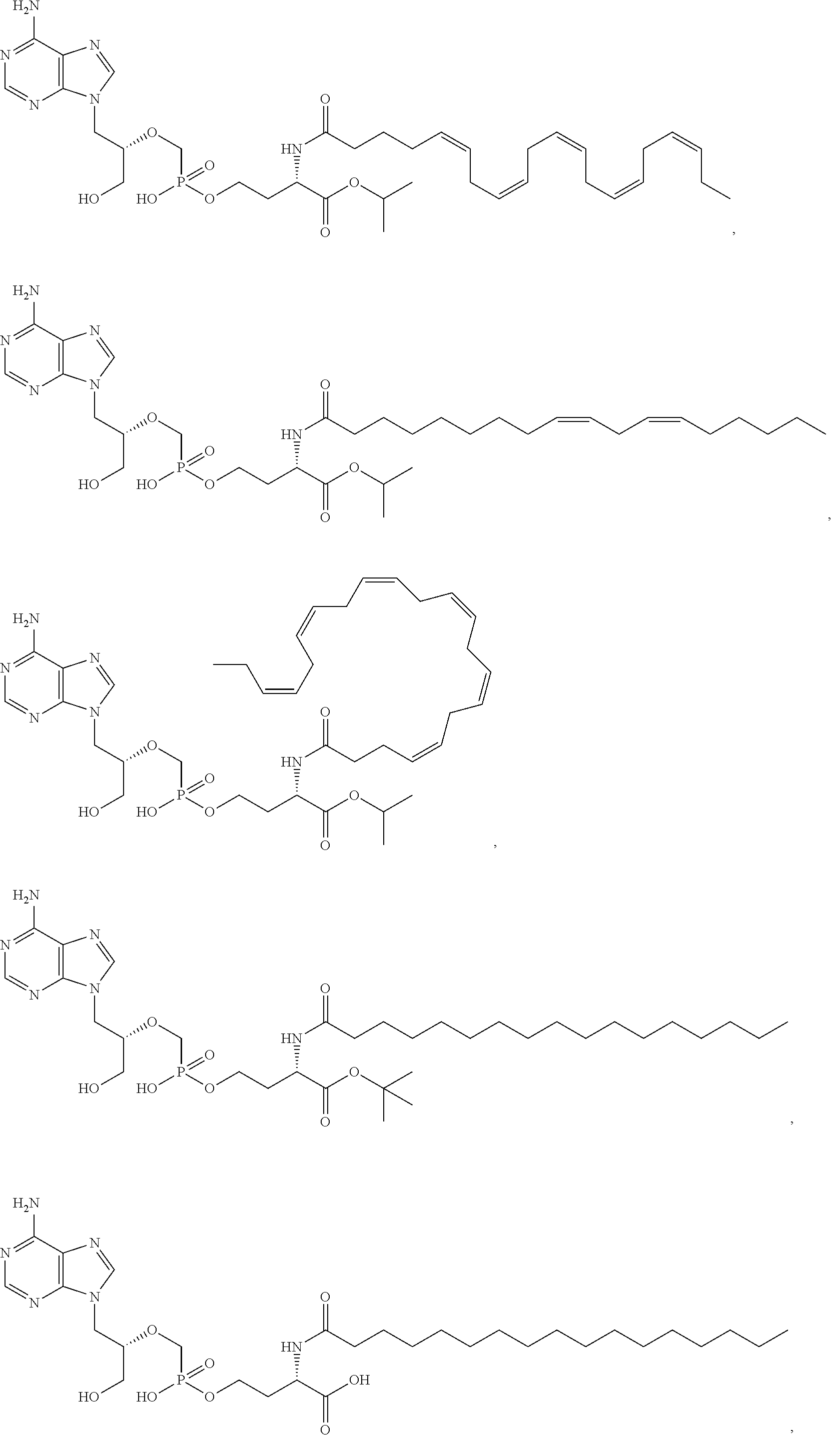 [SMILES: CC/C=C\C/C=C\C/C=C\C/C=C\C/C=C\CCCC(=O)N[C@@H](CCOP(=O)(O)CO[C@H](CO)Cn1cnc2c(N)ncnc21)C(=O)OC(C)C ; CCCCC/C=C\C/C=C\CCCCCCCC(=O)N[C@@H](CCOP(=O)(O)CO[C@H](CO)Cn1cnc2c(N)ncnc21)C(=O)OC(C)C ; CC/C=C\C/C=C\C/C=C\C/C=C\C/C=C\C/C=C\CCC(=O)N[C@@H](CCOP(=O)(O)CO[C@H](CO)Cn1cnc2c(N)ncnc21)C(=O)OC(C)C ; CCCCCCCCCCCCCCCCC(=O)N[C@@H](CCOP(=O)(O)CO[C@H](CO)Cn1cnc2c(N)ncnc21)C(=O)OC(C)(C)C ; CCCCCCCCCCCCCCCCC(=O)N[C@@H](CCOP(=O)(O)CO[C@H](CO)Cn1cnc2c(N)ncnc21)C(=O)O]

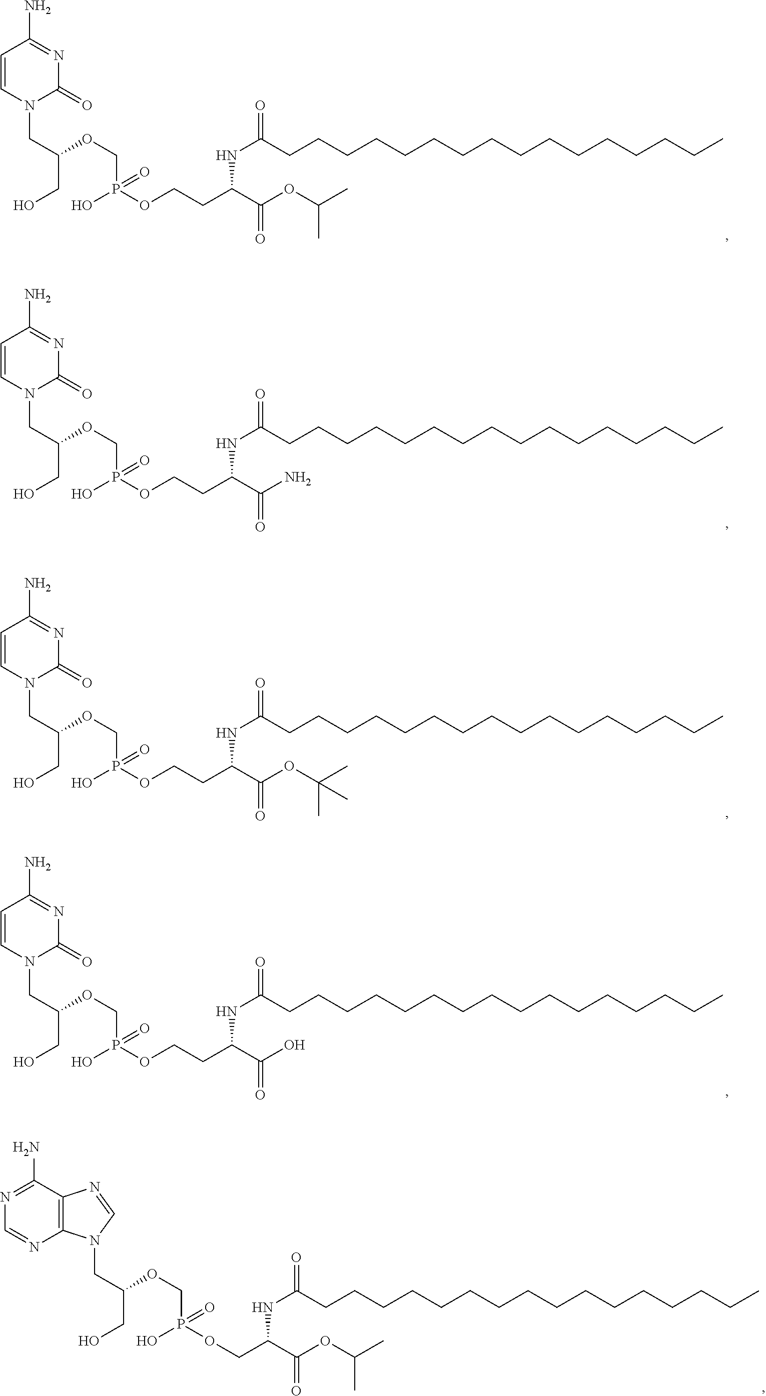 [SMILES: CCCCCCCCCCCCCCCCC(=O)N[C@@H](CCOP(=O)(O)CO[C@H](CO)Cn1ccc(N)nc1=O)C(=O)OC(C)C ; CCCCCCCCCCCCCCCCC(=O)N[C@@H](CCOP(=O)(O)CO[C@H](CO)Cn1ccc(N)nc1=O)C(N)=O ; CCCCCCCCCCCCCCCCC(=O)N[C@@H](CCOP(=O)(O)CO[C@H](CO)Cn1ccc(N)nc1=O)C(=O)OC(C)(C)C ; CCCCCCCCCCCCCCCCC(=O)N[C@@H](CCOP(=O)(O)CO[C@H](CO)Cn1ccc(N)nc1=O)C(=O)O ; CCCCCCCCCCCCCCCCC(=O)N[C@@H](COP(=O)(O)CO[C@H](CO)Cn1cnc2c(N)ncnc21)C(=O)OC(C)C]

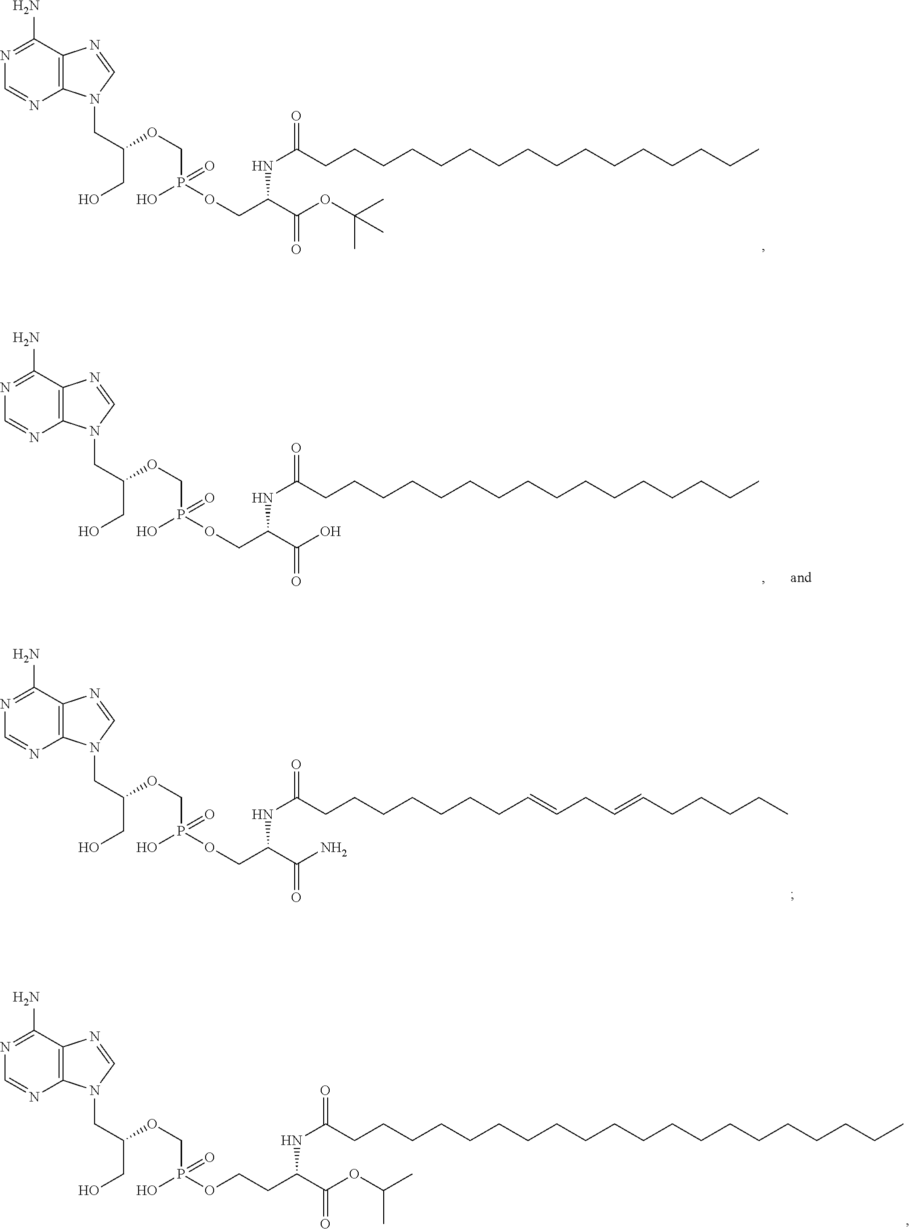 [SMILES: CCCCCCCCCCCCCCCCC(=O)N[C@@H](COP(=O)(O)CO[C@H](CO)Cn1cnc2c(N)ncnc21)C(=O)OC(C)(C)C ; CCCCCCCCCCCCCCCCC(=O)N[C@@H](COP(=O)(O)CO[C@H](CO)Cn1cnc2c(N)ncnc21)C(=O)O ; CCCCC/C=C/C/C=C/CCCCCCCC(=O)N[C@@H](COP(=O)(O)CO[C@H](CO)Cn1cnc2c(N)ncnc21)C(N)=O ; CCCCCCCCCCCCCCCCCCCCC(=O)N[C@@H](CCOP(=O)(O)CO[C@H](CO)Cn1cnc2c(N)ncnc21)C(=O)OC(C)C]

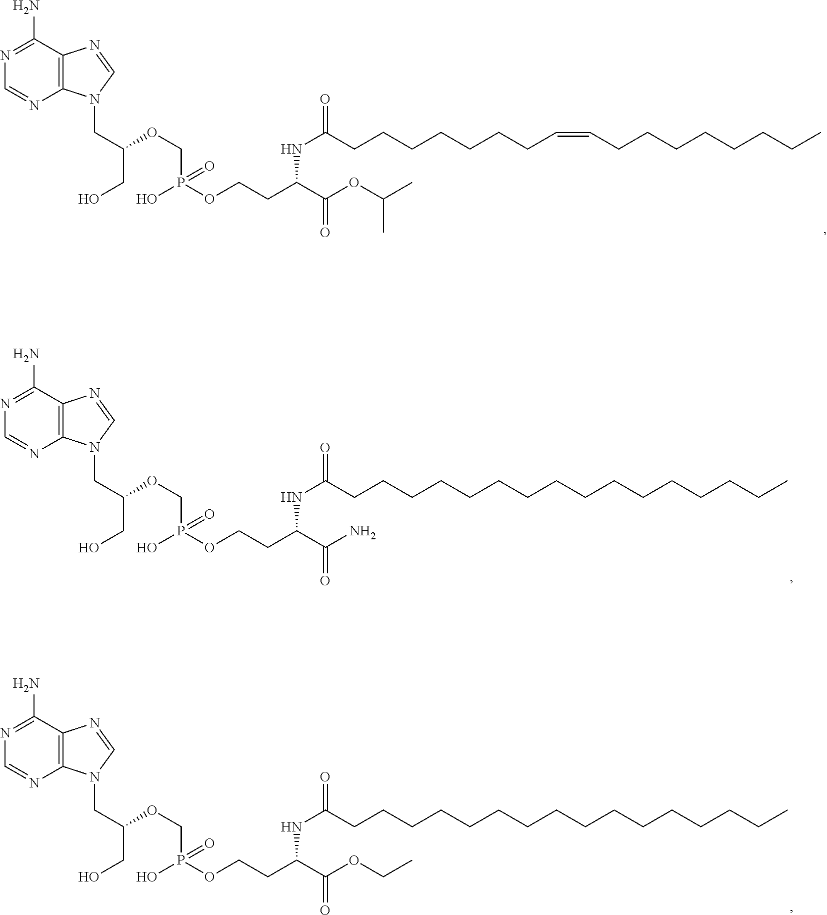 [SMILES: CCCCCCCC/C=C\CCCCCCCC(=O)N[C@@H](CCOP(=O)(O)CO[C@H](CO)Cn1cnc2c(N)ncnc21)C(=O)OC(C)C ; CCCCCCCCCCCCCCCCC(=O)N[C@@H](CCOP(=O)(O)CO[C@H](CO)Cn1cnc2c(N)ncnc21)C(N)=O ; CCCCCCCCCCCCCCCCC(=O)N[C@@H](CCOP(=O)(O)CO[C@H](CO)Cn1cnc2c(N)ncnc21)C(=O)OCC]

and pharmaceutically acceptable salts thereof.

In another aspect, D_{M} includes a phosphorus-containing drug moiety selected from the group consisting of phosphates, phosphonates, and phosphinates. In a refinement, D_{M} includes a phosphorus-containing drug moiety selected from the group consisting of phosphates and phosphonates. In a variation, D_{M} includes an acyclic nucleoside phosphonate selected from the group consisting of cidofovir, (S)-HPMPA, adefovir, tenofovir, and analogues thereof or a fragment thereof. In a refinement, the acyclic nucleoside phosphonate is cidofovir or(S)-HPMPA.

In another aspect, D_{M} includes a nucleoside monophosphate derived from a nucleoside selected from the group consisting of remdesivir, sofosbuvir, gemcitabine, cytarabine, fludarabine, cladribine, and clofarabine or a fragment thereof.

In another aspect, D_{M} includes a carboxylic acid-containing drug moiety.

In another aspect, the D_{M} is effective for treating a viral infection caused by a virus selected from the group consisting of adenovirus, coronavirus, cytomegalovirus, and varicella zoster virus.

In another aspect, the D_{M} is effective for treating cancer.

In a further aspect, a method of preparing a nucleoside phosphonate is provided. The method includes selective dealkylation of a mixed phosphonate monoalkyl diester by bromotrimethylsilane (BTMS), where: B is a natural or unnatural purine or pyrimidine base; R is H or C(O)R₂, wherein R₂ is lipid-like; and R₁ is NHR₁ₐ, where R₁ₐ is H or lipid-like.

In some embodiments of the method: a) B is cytosine or adenine; b) R₂ is alkyl, which can be C₈₋₁₈ alkyl; c) R₁ₐ is alkyl, alkene, ether or thioether, which can be C₁₂₋₁₈ alkyl, (CH₂)ₙCH═CHCₙ₁, (CH₂)ₙ—O—(CH₂)ₙ₁ or (CH₂)ₙ—S—(CH₂)ₙ₁ wherein n and/or n1=4-9; d) the CH₂Ph group can be replaced by CH₂, CH₂CH₂, or CHCH₃; or any combination of a)-d)

In a further aspect, a method of preparing a nucleoside phosphate is provided. The method includes CDI conjugation of a phosphorylated amino acid with pyrophosphate or pyrophosphate analogues, where B is a purine or pyrimidine base; R is H or C(O)NHR₂, wherein R₂ is lipid-like; and R₁ is NHR₁ₐ, where R₁ₐ is H or lipid-like.

In some embodiments of the method: a) B is cytosine, guanine, adenine or thymine, or a related structure; b) R₂ is alkyl, which can be C₈₋₁₈ alkyl; c) R₁ₐ is alkyl, alkene, ether or thioether, which can be C₁₂₋₁₈ alkyl, (CH₂)ₙCH═CHCₙ₁, (CH₂)ₙ—O—(CH₂)ₙ₁ or (CH₂)ₙ—S—(CH₂)ₙ₁ wherein n and/or n1=4-9; d) the CH₂Ph group can be replaced by CH₂, CH₂CH₂, or CHCH₃; e) Y is O, NH, CH₂, CHF, CHCl, CHBr, CF₂, CCl₂, CBr₂, CCH₃, C(CH₃)₂, CHN₃, or CCH₃N₃; f) Z is a ribose or deoxy ribose sugar, or a related structure; m=0-1; m₁=0-3; m₂=0-3; or any combination of a)-f).

In another aspect, Homoserinamide prodrugs of(S)-HPMPA have been synthesized and evaluated for antiviral activity. USC-093, an L-homoserinamide(S)-HPMPA prodrug, and USC-093D, a D-homoserinamide(S)-HPMPA prodrug, were evaluated against HAdV-C5 and HAdV-C6 in human foreskin fibroblast (HFF) cells. USC-093D exhibited EC50 values of 30 nM against HAdV-C5 and 1 nM against HAdV-C6, whereas USC-093 exhibited EC50 values of 70 nM against HAdV-C5 and 6 nM against HAdV-C6. These prodrugs were several orders of magnitude more potent than the positive control CDV (EC50 2000 nM against HAdV-C5 and 500 nM against HAdV-C6). The D-homoserinamide stereoisomer USC-093D demonstrated enhanced potency compared to the L-isomer USC-093, indicating that the stereochemistry of the promoiety can influence antiviral activity.

USC-150, an L-homoserinamide prodrug of HPMPC (CDV), has demonstrated significant therapeutic efficacy against HAdV-C6 in Syrian hamster models. In prophylactic studies, USC-150 significantly suppressed HAdV-C6 replication in the liver of intravenously infected Syrian hamsters, preventing mortality and reducing weight loss and liver damage, with efficacy observed at doses as low as 0.6 mg/kg. In therapeutic studies using hamsters intravenously infected with a lethal dose (2×10¹⁰ PFU/kg) of HAdV-C6, oral administration of USC-150 at 5 mg/kg once daily reduced virus replication in the liver to non-detectable or non-quantifiable levels and largely prevented viral pathogenesis when treatment started at 2 days after virus challenge or earlier. Even when treatment was delayed until 3 days post challenge, USC-150 reduced virus replication and significantly mitigated HAdV-C6-induced pathology. Untreated animals experienced rapid weight loss and high mortality, while those receiving USC-150 showed mitigated weight loss and survival benefits, with recovered animals regaining weight at rates comparable to uninfected controls.

USC-374, an(S)-HPMPA tyrosinamide prodrug, has demonstrated potent antiviral activity against HAdV-C6 in cell culture and in Syrian hamster models. In studies comparing routes of administration, USC-374 was administered orally (p.o.) or subcutaneously (s.c.) at 10 mg/kg once daily to immunosuppressed Syrian hamsters, starting one day before intranasal infection with 1×10¹⁰ PFU/kg HAdV-C6. Subcutaneous administration of USC-374 significantly outperformed oral administration, reducing lung inflammation, lowering lung viral loads, and mitigating weight loss in the infected animals. These results indicate that subcutaneous administration of USC-374 provides an effective strategy to reduce premature metabolism, greatly enhancing its effectiveness in reducing lung pathology caused by adenovirus infection.

In embodiments that include a method of inhibiting viral replication or a method of treating a virus infection, the virus may be an RNA virus, a DNA virus, or a retrovirus, for example. Particular examples of viruses include, but are not limited to, severe acute respiratory syndrome coronavirus 2 (SARS-COV-2), varicella zoster virus (VZV), human papillomavirus (HPV), cytomegalovirus (CMV).

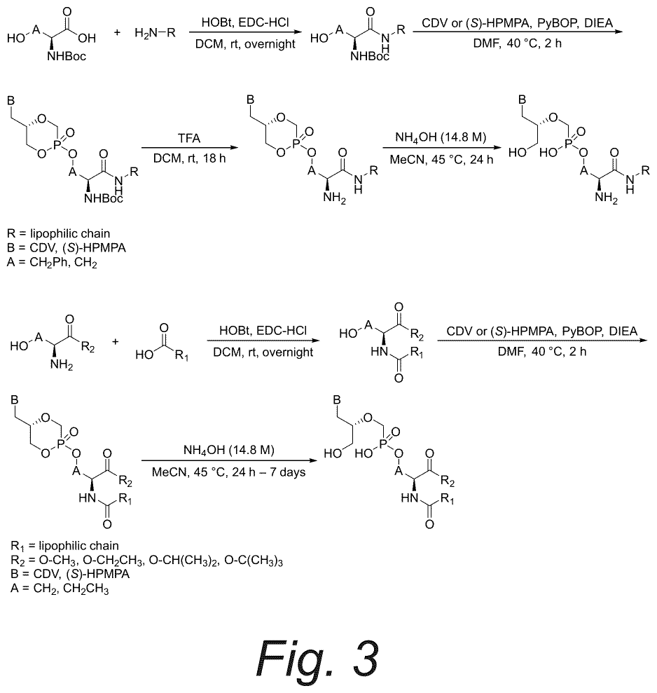

FIG. 3 provides a synthetic scheme for making compounds of the present disclosure.

The present invention may be better understood by referring to the accompanying examples, which are intended for illustration purposes only and should not in any sense be construed as limiting the scope of the invention.

### Example 1

### Synthesis of CDV and HPMPA Prodrugs

### Example 1a. General Synthesis of Novel Prodrugs

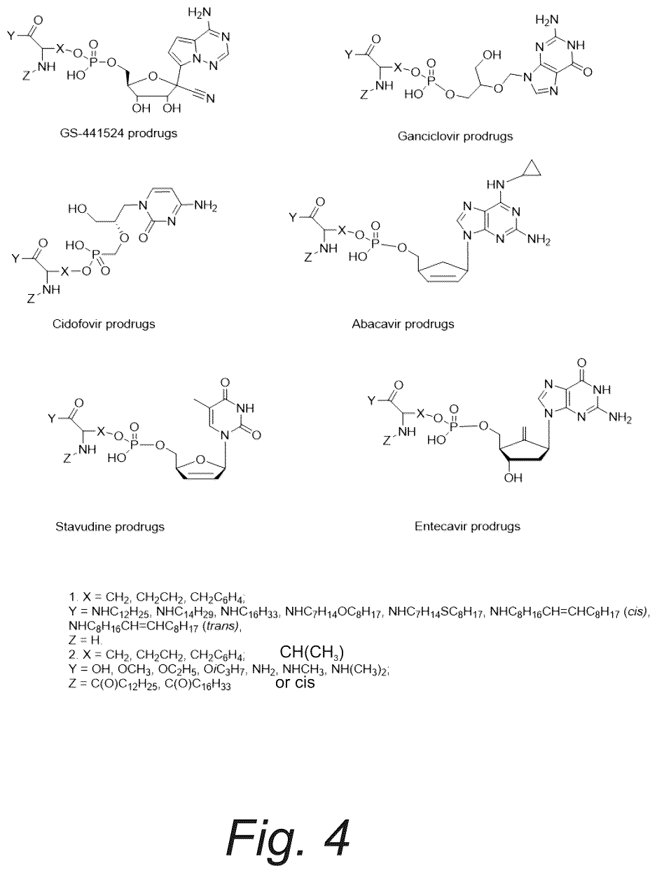

The structures of embodiments of this invention are shown in FIG. 4 (Series A-D). The structural features that are changed to modify biological and pharmacological properties of the prodrugs: i) lipophilic chain length and structure (Series A); by introducing unsaturation at the center of the N-alkyl group; ii) introduction of a polar atom (O or S) at the center of the lipid modifier to create a site for enzymatic cleavage to decrease lipidomimetic properties of the prodrug in the cell prior to P—O ester cleavage or a means to increase prodrug aqueous solubility without reducing the chain length with only a modest (ΔpD˜−1) impact on lipophilicity (Series B); iii) variation in the position of the N-alkyl chain by moving it to the amino side of the promoiety (Series C); this modification converts the prodrug from a zwitterion (more lipidomimetic) to a monoamine PO— (more soluble in aqueous media); iv) the amino acid P—O ester linkage (O-Tyr, O-Ser, O-Homo-Ser or O-Thr) to vary metabolic activation and other PK properties to tune enzyme-dependent activation of the prodrug by cellular phospholipases/phosphatases (Series D). The person skilled in the art will appreciate that by some of the modifications taught in this invention or similar ones, it is possible to modulate the overall charge on the prodrug molecule and specifically, its promoiety to advantage. For example, introduction of a positive charge by retention of the free amino group and neutralization of the remaining negative charge of the esterified phosphonic acid group by conversion to a cyclic, amido or simple alkyl or aryl ester forms.

### Experimental

### General Syntheses of the Lipidomimetic Synthons: Commercially Unavailable N-Alkyl Promoieties can be Synthesized by Literature Methods (30-32).

Conjugation of the lipidomimetic synthons to(S)-HPMPC and(S)-HPMPA: Prodrugs that incorporate a derivatized single amino acid having a hydroxy side chain in the promoiety portion of the molecule can be prepared following the generalized synthetic pathway shown in FIG. 3 (33). The conjugation of the amino acid promoieties to CDV (or HPMPA) via an internal phosphoester bond in the penultimate synthetic step produces a new chiral center at phosphorus (Sp or Rp); however, these cyclic NP intermediates, generated as diastereomeric mixtures, are converted to the same final nucleoside phosphonate analogue in the last step. (The cyclic phosphonate intermediates are themselves active prodrugs (34) but have different metabolic t½ and require an additional purification step for individual isolation. They are considered as further embodiments of this invention, providing a neutral phosphonate group in the final prodrug structure, which may be advantageous as explained in the preceding paragraph. The tBoc-protected amino acid synthons can be prepared by a previously described method (33). Alkenyl lipophilic substituent (Series A) precursors are advantageously 1-aminoalkenes (cis or trans) which can be added to N-tBoc-protected amino acids by reaction with HOBt/EDC in DCM at 25° C. Alkoxyalkyl lipophilic substituents (Series B) can be prepared as follows: 1) (example, C8-O-C8): 8-bromo-1-octanal is converted to the phthalimide alcohol in DMF, then reacted with 1-bromooctane and NaH in DMF, and deprotected with hydrazine in EtOH; 2) (example, C8-S—C8): 1,8-dibromooctane is similarly monoprotected, then reacted with 1-bromooctane and NaOH in thiourea.

### Example 1b. Large-Scale Syntheses of Analogues

For large-scale synthesis, the known chemoselectivity of BTMS-silyldealkylation (35, 36) for alkyl (i.e., ethyl or methyl) vs. aryl (i.e., tyrosinyl) or sterically larger than methyl (i.e. serinyl, homoserinyl, threonyl)phosphonate esters can be utilized. Elimination of the NH4OH hydrolysis step in FIG. 3, which typically limits the overall yield (˜50%), makes this method attractive for further scaling development. An important feature of this new method is that it allows the intermediates to be purified by silica gel column chromatography, avoiding preparative HPLC which may be impractical and too time-consuming for large-scale synthesis. After BTMS deprotection, the final products can also be isolated by crystallization, which is highly scalable.

### Example 2

### Synthesis and Evaluation of USC-093 and USC-093D

USC-093, an L-homoserinamide(S)-HPMPA prodrug, and USC-093D, a D-homoserinamide(S)-HPMPA prodrug, were synthesized from Boc-protected L-homoserine or D-homoserine lactone, respectively. The two stereoisomeric prodrugs were evaluated for antiviral activity in vitro against HAdV-C₅ and HAdV-C₆ in human foreskin fibroblast (HFF) cells and A549 cells. Cidofovir (CDV) was used as the positive control.

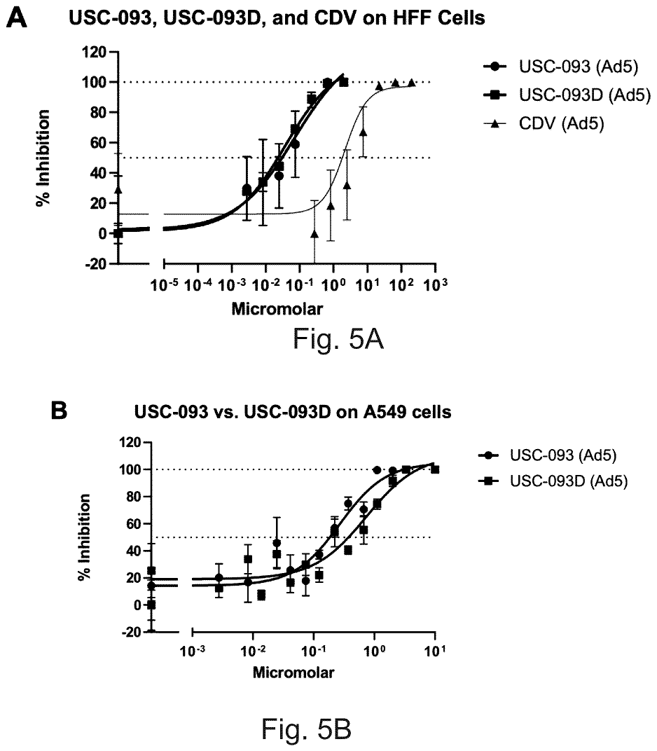

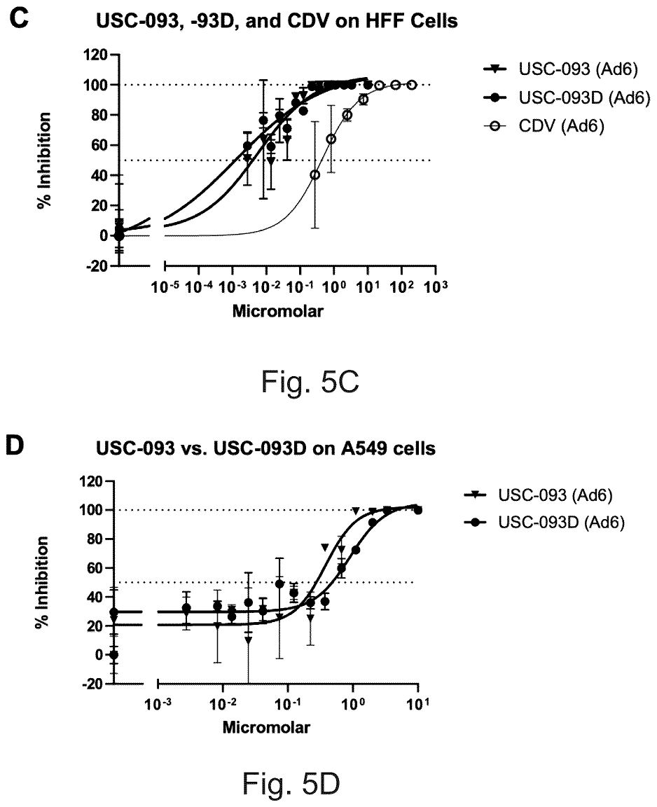

In HFF cells, USC-093D exhibited EC50 values of 30 nM against HAdV-C5 and 1 nM against HAdV-C6. USC-093 exhibited EC50 values of 70 nM against HAdV-C5 and 6 nM against HAdV-C6. CDV exhibited EC50 values of 2000 nM against HAdV-C5 and 500 nM against HAdV-C6. Both prodrugs were several orders of magnitude more potent than CDV (FIG. 5). The D-homoserinamide stereoisomer USC-093D demonstrated approximately 2-fold to 6-fold enhanced potency compared to the L-isomer USC-093.

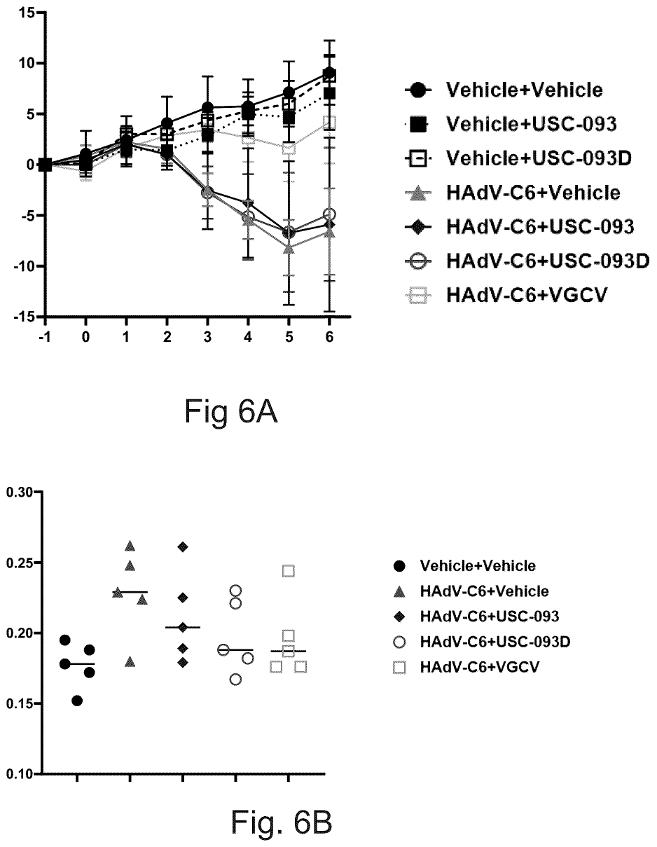

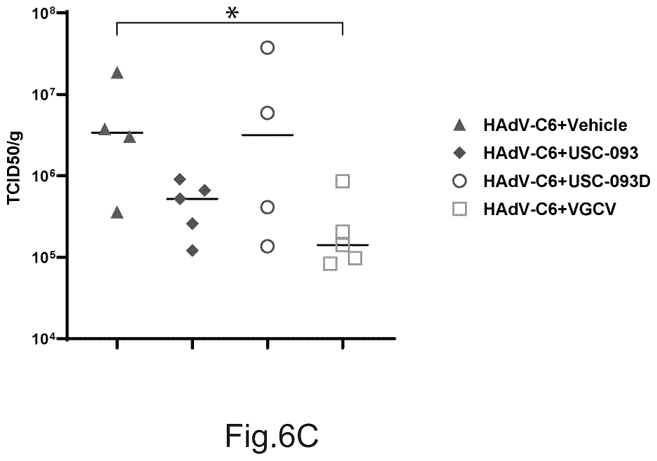

USC-093D and USC-093 were also compared for efficacy in a Syrian hamster model. Hamsters were immunosuppressed with intraperitoneal (i.p.) injections of cyclophosphamide (140 mg/kg initial dose followed by 100 mg/kg twice-weekly doses). The hamsters were instilled intranasally (i.n.) with vehicle or 4×10¹⁰ PFU/kg of HAdV-C6. Drug administration started 1 day before challenge and continued for the duration of the study (20 mg/kg p.o. once daily). Valganciclovir (VGCV) at 200 mg/kg twice daily p.o. was used as the positive control. At 3 days post challenge, lung samples were collected, and the virus burden was determined by TCID50 assay. At the dose of 20 mg/kg oral administration, the lung weights were marginally lower in the USC-093D-treated group compared to the HAdV-C6+Vehicle group. USC-093 marginally inhibited the virus replication in the lungs of HAdV-C6-infected hamsters (FIG. 6).

### Example 3

### Synthesis and Evaluation of USC-150

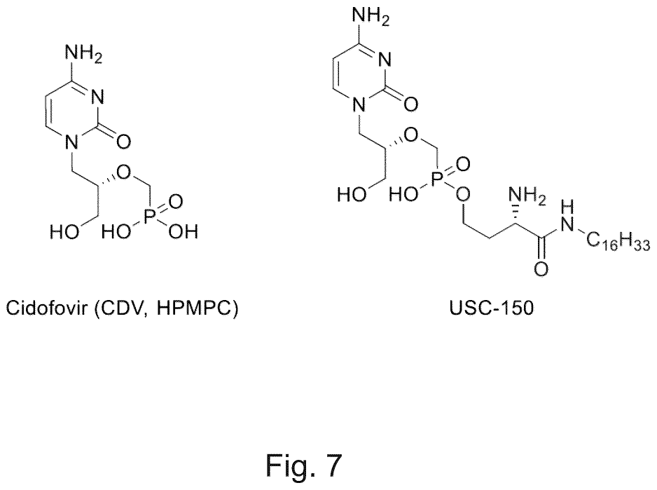

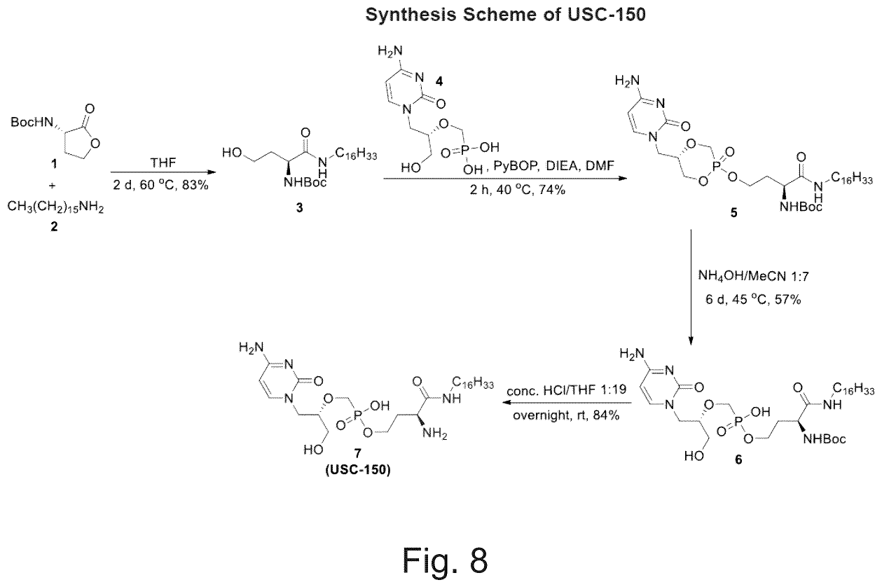

USC-150, an L-homoserinamide prodrug of HPMPC (CDV) (FIG. 7), was synthesized in a total of four steps (FIG. 8). The synthesis involved: (1) reaction of Boc-protected homoserine lactone with hexadecylamine in THF at 60° C. (83% yield); (2) coupling with CDV using PyBOP, DIEA in DMF at 40° C. (74% yield); (3) deprotection with conc. HCl/THF at room temperature (84% yield); and (4) ring opening with aqueous NH₄OH/MeCN (57% yield).

The therapeutic efficacy of USC-150 was evaluated in Syrian hamsters. Hamsters were immunosuppressed using cyclophosphamide (140 mg/kg initial dose followed by 100 mg/kg twice-weekly doses) via intraperitoneal (i.p.) injections. Virus was injected intravenously at a dose of 2×10¹⁰ PFU/kg. USC-150 was administered at 5 mg/kg p.o. once daily (q.d.), starting at 1, 2, or 3 days after HAdV-C6 injection. At 7 days post challenge, five hamsters were sacrificed, and serum and liver were collected. Virus burden in the liver and transaminase levels were determined.

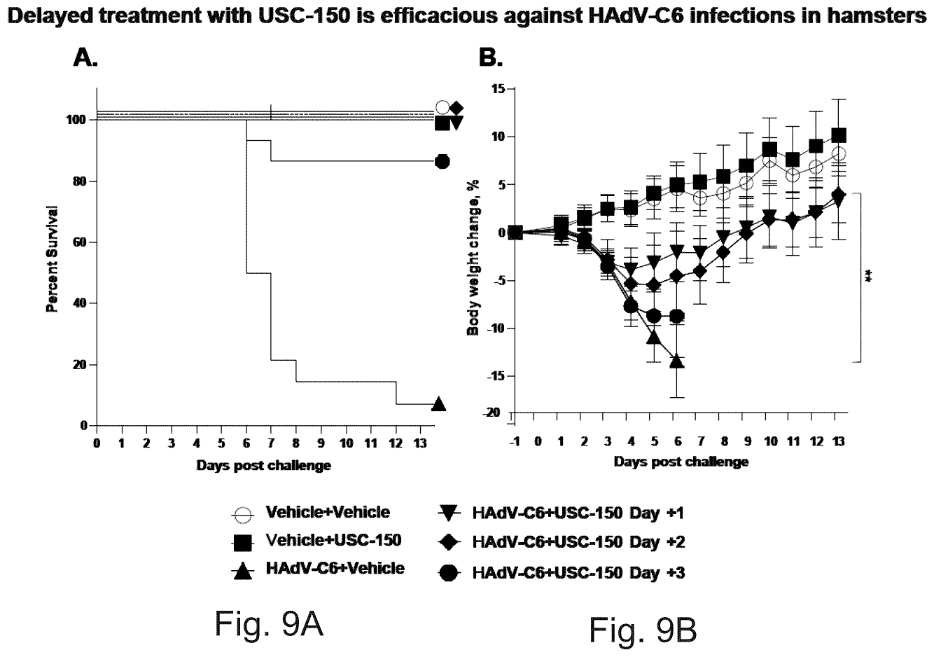

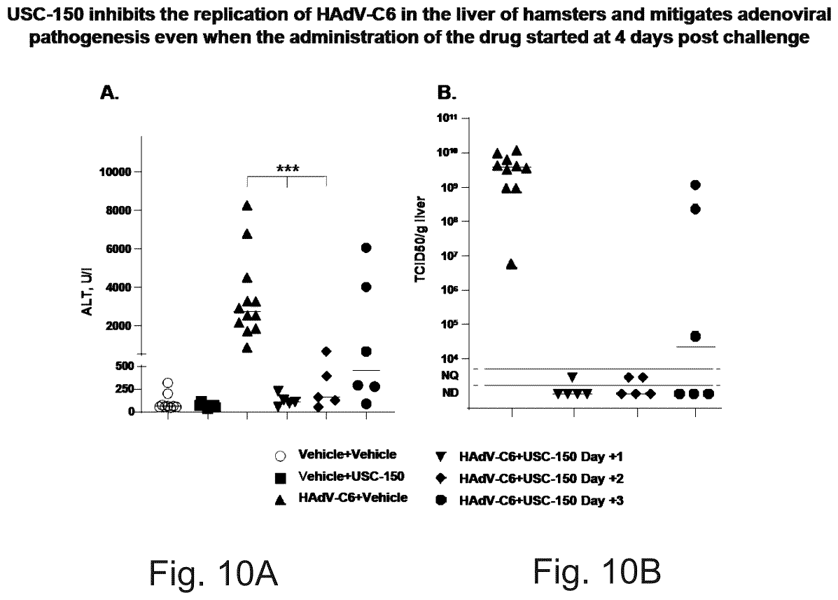

The 5 mg/kg p.o. q.d. dose of USC-150 reduced virus replication to non-detectable or non-quantifiable levels and largely prevented viral pathogenesis when treatment started at 2 days after virus challenge or earlier. Even when treatment was delayed until 3 days post challenge, USC-150 reduced virus replication and significantly mitigated HAdV-C6-induced pathology. Survival analysis showed that HAdV-C6+Vehicle versus all USC-150-treated groups had p<0.0001 (log rank test) (FIG. 9). Serum alanine transaminase levels were significantly reduced in USC-150-treated groups (p<0.001, two-tailed Mann-Whitney test) (FIG. 10). Untreated animals experienced rapid weight loss and high mortality, while those receiving USC-150 showed mitigated weight loss and survival benefits.

### Example 4

### Synthesis and Evaluation of USC-374

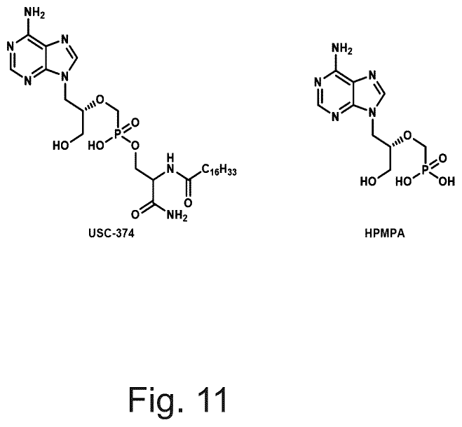

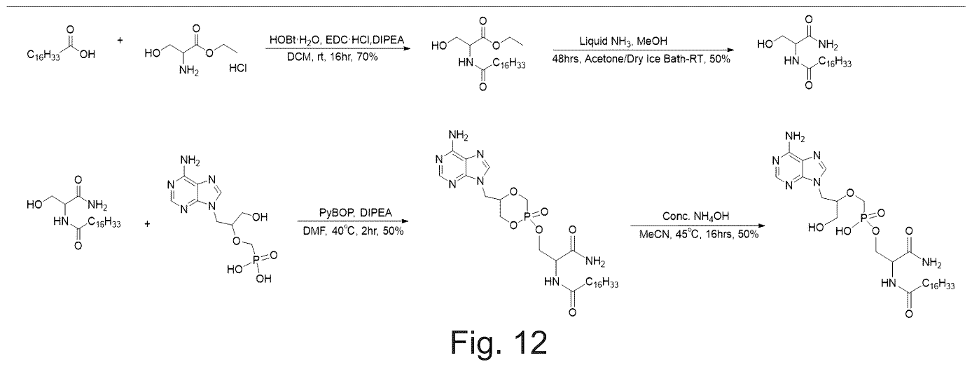

USC-374, an(S)-HPMPA tyrosinamide prodrug (FIG. 11), was synthesized on gram scale (FIG. 12). The synthesis proceeded via coupling of the appropriately protected tyrosine promoiety with(S)-HPMPA followed by deprotection steps.

The efficacy of USC-374 was evaluated in immunosuppressed Syrian hamsters with intranasal HAdV-C6 infection. Hamsters were immunosuppressed with intraperitoneal (i.p.) injections of cyclophosphamide (140 mg/kg initial dose followed by 100 mg/kg twice-weekly doses). The hamsters were intranasally instilled with vehicle or 1×10¹⁰ PFU/kg of HAdV-C6. USC-374 was administered orally (p.o.) or subcutaneously (s.c.) at 10 mg/kg q.d., starting one day before HAdV-C6 infection and continued for the duration of the study. Valganciclovir (VGCV) at 200 mg/kg p.o. twice daily (b.i.d.) was used as the positive control.

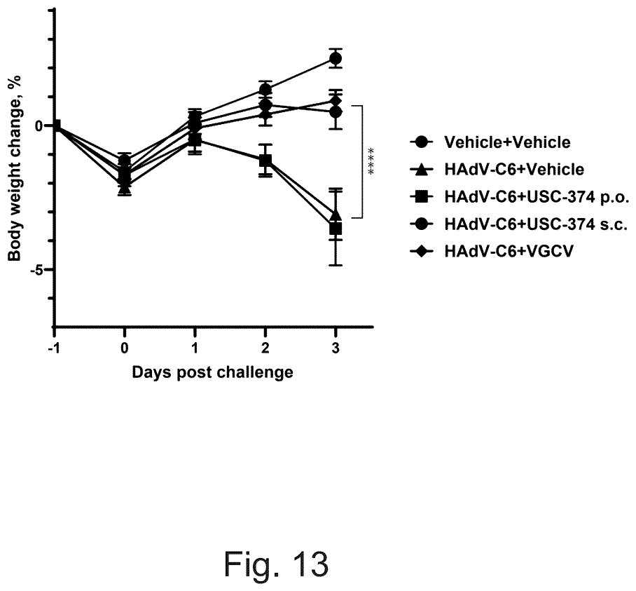

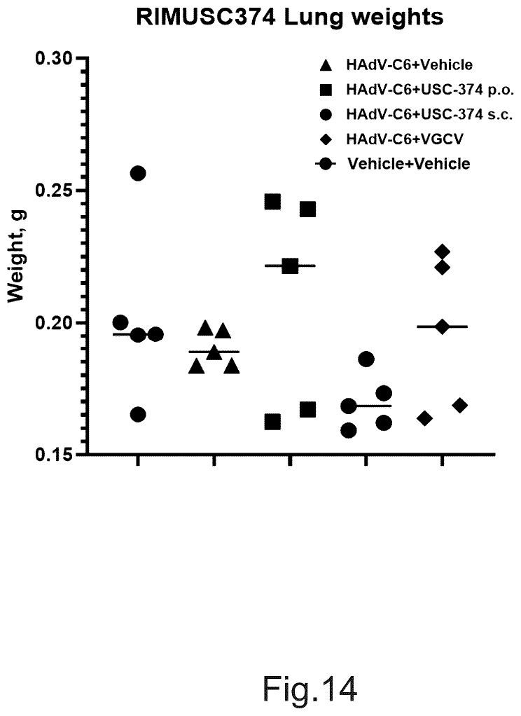

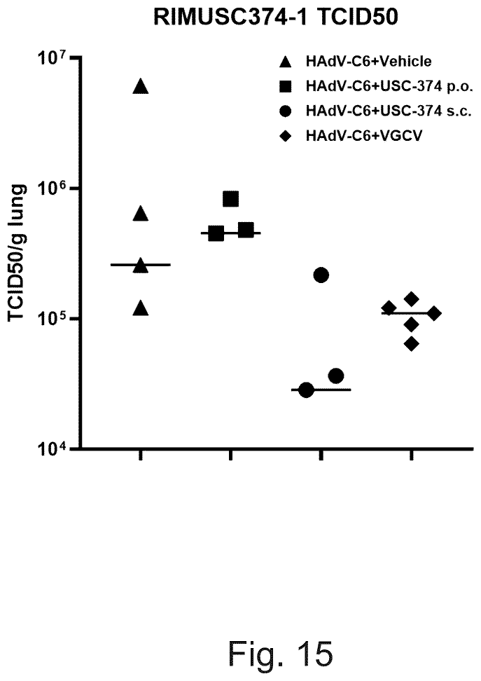

Subcutaneous administration of USC-374 significantly outperformed oral administration. The HAdV-C6+USC-374 s.c. group exhibited significantly less weight loss compared to the HAdV-C6+Vehicle group by day 3 (p<0.001, two-way ANOVA) (FIG. 13). Lung weights, as a surrogate for viral-induced inflammation severity, were lower in the low-dose (10 mg/kg) USC-374 s.c. group than in virus-only controls (p<0.05, Mann-Whitney U-test). At day 3 post-challenge, subcutaneous administration of USC-374 (10 mg/kg) lowered lung viral titers compared to untreated controls (p=0.057, Mann-Whitney U), despite being administered at a much lower dose than the VGCV positive control (200 mg/kg) (FIGS. 14 and 15). These results indicate that subcutaneous administration of USC-374 provides an effective strategy to reduce premature metabolism, greatly enhancing its effectiveness in reducing lung pathology caused by adenovirus infection.

### Example 5. Synthesis of USC-636

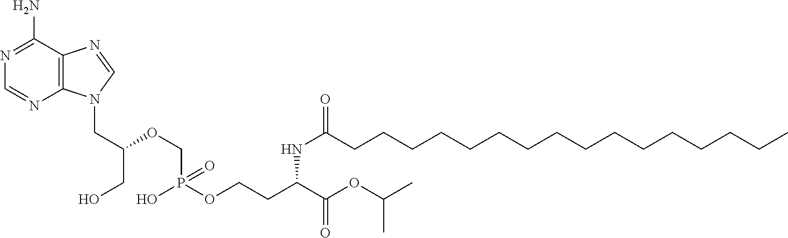 [SMILES: CCCCCCCCCCCCCCCCC(=O)N[C@@H](CCOP(=O)(O)CO[C@H](CO)Cn1cnc2c(N)ncnc21)C(=O)OC(C)C]

Thionyl chloride (5.75 eq, 24.14 mmol) was slowly added to isopropanol (14 mL) at 0° C. After the reaction mixture was warmed to room temperature, L-homoserine (1 eq, 4.2 mmol) was added, and the mixture was refluxed for 8 h. The reaction mixture was concentrated under reduced pressure, and the residue was purified by automated flash chromatography using a gradient of ethyl acetate/methanol to afford isopropyl L-homoserinate (21%).

Isopropyl L-homoserinate (1 eq, 0.38 mmol) and heptadecanoic acid (1.1 eq, 0.42 mmol) were dissolved in dichloromethane (0.1 M, 4 mL), followed by the addition of 1-hydroxybenzotriazole hydrate (1.2 eq, 0.46 mmol) and 1-ethyl-3-(3-dimethylaminopropyl) carbodiimide hydrochloride: (1.2 eq, 0.46 mmol). N,N-Diisopropylethylamine (3 eq, 1.14 mmol) was then added, and the reaction mixture was stirred at room temperature overnight. Upon completion, the mixture was diluted with dichloromethane (10 mL) and washed sequentially with 1.6 M citric acid, saturated aqueous sodium bicarbonate, and brine. The organic layer was dried over anhydrous sodium sulfate and concentrated under reduced pressure. The residue was purified by automated flash chromatography on silica using a gradient of hexane/ethyl acetate to yield isopropyl heptadecanoyl-L-homoserinate (68%).

(S)-HPMPA (1 eq, 0.093 mmol) and isopropyl heptadecanoyl-L-homoserinate (1.3 eq, 0.12 mmol) were dissolved in N,N-dimethylformamide (0.12 M, 0.8 mL). N,N-Diisopropylethylamine (19 eq, 1.77 mmol) and benzotriazol-1-yl-oxytripyrrolidinophosphonium hexafluorophosphate (4.3 eq, 0.40 mmol) were added, and the reaction mixture was stirred at 40° C. for 2 h. The solvent was removed under reduced pressure, and the crude product was purified by automated flash chromatography on silica using a gradient of dichloromethane/methanol to afford isopropyl O-((5S)-5-((6-amino-9H-purin-9-yl)methyl)-2-oxido-1,4,2-dioxaphosphinan-2-yl)-N-heptadecanoyl-L-homoserinate (66%).

The resulting dioxaphosphinane intermediate (1 eq, 0.079 mmol) was dissolved in acetonitrile (16 mL, 5 mM), followed by the addition of 14.8 M ammonium hydroxide (394 eq, 31.12 mmol). The reaction mixture was heated at 45° C. for 7 days. After completion, the solvent was removed under reduced pressure, and the residue was purified by flash chromatography on silica using a gradient of dichloromethane/methanol to yield ((((S)-1-(6-amino-9H-purin-9-yl)-3-hydroxypropan-2-yl)oxy)methyl)((S)-4-heptadecanamido-5-isopropoxy-5-oxopentyl)phosphinic acid (70%), (USC-636).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A prodrug comprising a compound having formula (1): 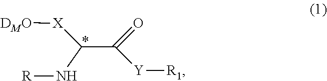
or a pharmaceutically acceptable salt thereof,
wherein:
D_{M} is a drug moiety;
X is selected from the group consisting of C₁-C₄ alkylene, C₆-C₁₀ arylene, and C₇-C₁₂ alkylarylene;
Y is O or NH;
R is H, C₁-C₆ alkyl, or C(O)R₂;
R₁ is H, C₁-C₂₅ alkyl, C₂-C₂₅ alkenyl, C₂-C₂₅ alkoxyalkyl, or C₂-C₂₅ alkylthioalkyl; and
R₂ is lipid-like.

2. The prodrug of claim 1, wherein R, R₁, and R₂ are each independently C₅ to C₂₅ alkyl, C₅ to C₂₅ alkylene, or C₁₀ to C₂₆ aryl.

3. The prodrug of claim 1, wherein X is CH₂(C₆H₄), CH₂, CH₂CH₂, or CHCH₃.

4. The prodrug of claim 1, wherein D_{M} comprises a phosphorus-containing drug moiety selected from the group consisting of phosphates, phosphonates, and phosphinates.

5. The prodrug of claim 1, wherein D_{M} comprises an acyclic nucleoside phosphonate or fragment thereof selected from the group consisting of cidofovir, (S)-HPMPA, adefovir, tenofovir, and analogues thereof.

6. The prodrug of claim 5, wherein the acyclic nucleoside phosphonate is cidofovir.

7. The prodrug of claim 5, wherein the acyclic nucleoside phosphonate is(S)-HPMPA.

8. The prodrug of claim 1, wherein D_{M} comprises a nucleoside monophosphate or fragment thereof derived from a nucleoside selected from the group consisting of remdesivir, sofosbuvir, gemcitabine, cytarabine, fludarabine, cladribine, and clofarabine.

9. The prodrug of claim 1, wherein D_{M} comprises a carboxylic acid-containing drug moiety.

10. The prodrug of claim 1, wherein D_{M} is effective for treating a viral infection caused by a virus selected from the group consisting of adenovirus, coronavirus, cytomegalovirus, and varicella zoster virus.

11. The prodrug of claim 1, wherein D_{M} is effective for treating cancer.

12. The prodrug of claim 1, wherein D_{M} is 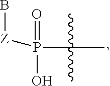
B is a purine base, pyrimidine base, or derivative thereof;
Z is a divalent moiety derived from a sugar, a cyclic sugar mimic, or an acyclic sugar mimic, wherein Z comprises a polyhydroxylated hydrocarbon chain.

13. The prodrug of claim 12, wherein Z is

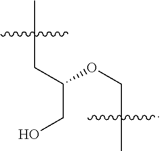 [SMILES: *CO[C@@H](C*)CO]

14. The prodrug of claim 1, wherein D_{M} is

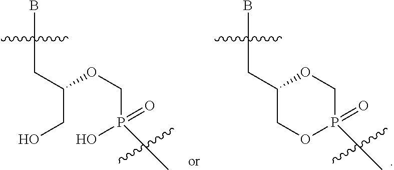

15. The prodrug of claim 1, wherein D_{M}O is: 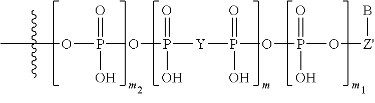
wherein
B is a purine base, pyrimidine base, or derivative thereof;
Z′ is a sugar, a cyclic sugar mimic or acyclic sugar mimic;
Y is O, NH, CH₂, CHF, CHCl, CHBr, CF₂, CCl₂, CBr₂, CCH₃, C(CH₃)₂, CHN₃, CCH₃N₃
m is 0 to 1;
m₁ is 0 to 3; and
m₂ is 0 to 3.

16. The prodrug of claim 15, wherein Z′ is

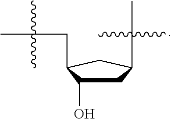

17. The prodrug of claim 15, wherein R is

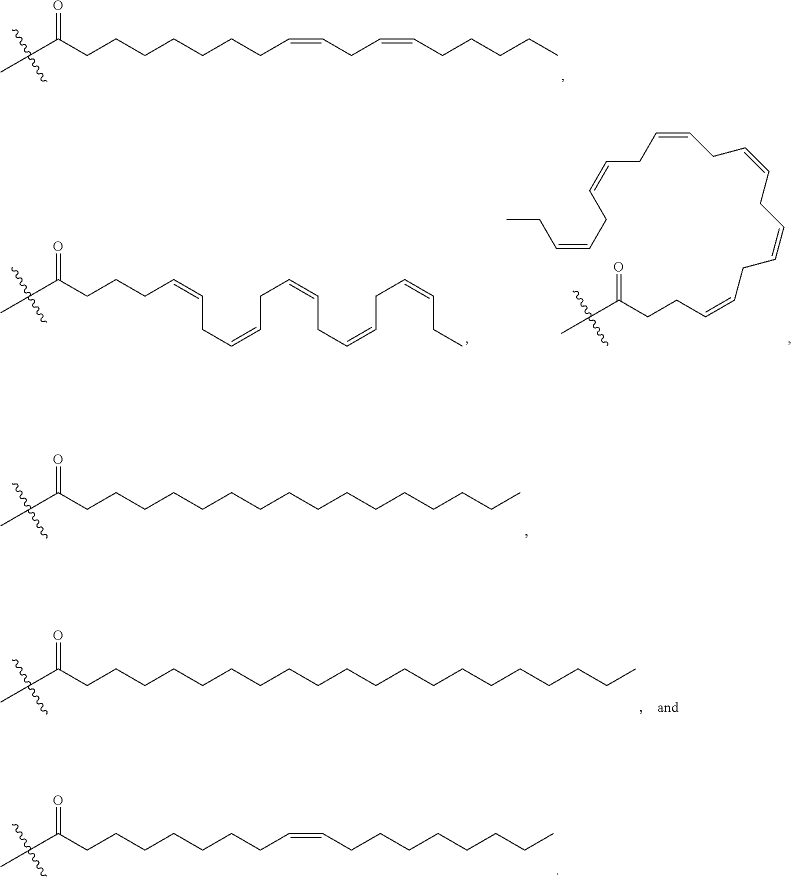

18. The prodrug of claim 15, wherein X is O and Ri is H or C₁-C₄ alkyl.

19. The prodrug of claim 15, wherein X is NH and R₁ is H or C₁-C₄ alkyl.

20. The prodrug of claim 1, wherein the carbon labeled with * provides an amino acid or amino acid derivative in the D form or the L form.

21. A compound selected from the group consisting of: 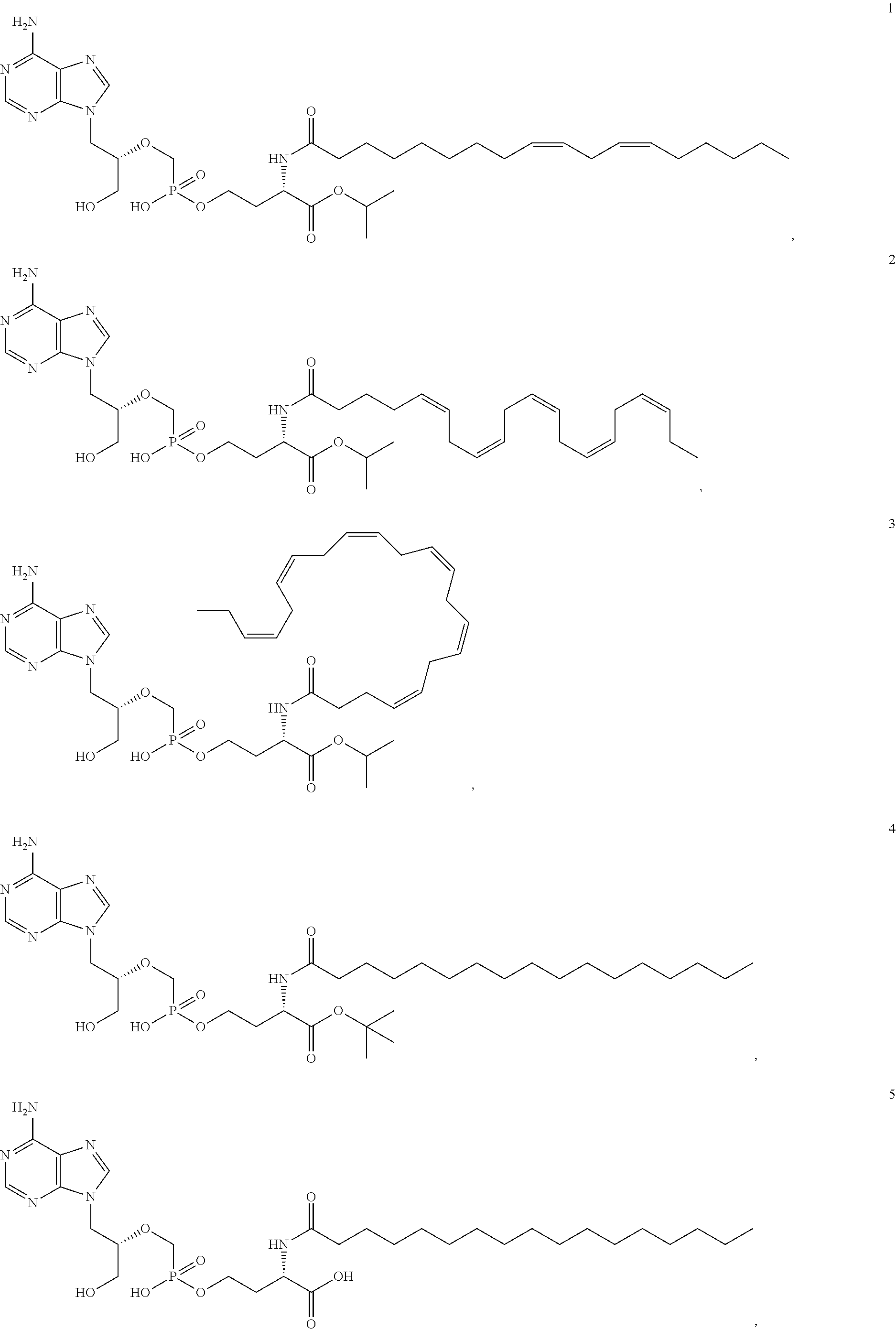 [SMILES: CCCCC/C=C\C/C=C\CCCCCCCC(=O)N[C@@H](CCOP(=O)(O)CO[C@H](CO)Cn1cnc2c(N)ncnc21)C(=O)OC(C)C ; CC/C=C\C/C=C\C/C=C\C/C=C\C/C=C\CCCC(=O)N[C@@H](CCOP(=O)(O)CO[C@H](CO)Cn1cnc2c(N)ncnc21)C(=O)OC(C)C ; CC/C=C\C/C=C\C/C=C\C/C=C\C/C=C\C/C=C\CCC(=O)N[C@@H](CCOP(=O)(O)CO[C@H](CO)Cn1cnc2c(N)ncnc21)C(=O)OC(C)C ; CCCCCCCCCCCCCCCCC(=O)N[C@@H](CCOP(=O)(O)CO[C@H](CO)Cn1cnc2c(N)ncnc21)C(=O)OC(C)(C)C ; CCCCCCCCCCCCCCCCC(=O)N[C@@H](CCOP(=O)(O)CO[C@H](CO)Cn1cnc2c(N)ncnc21)C(=O)O] 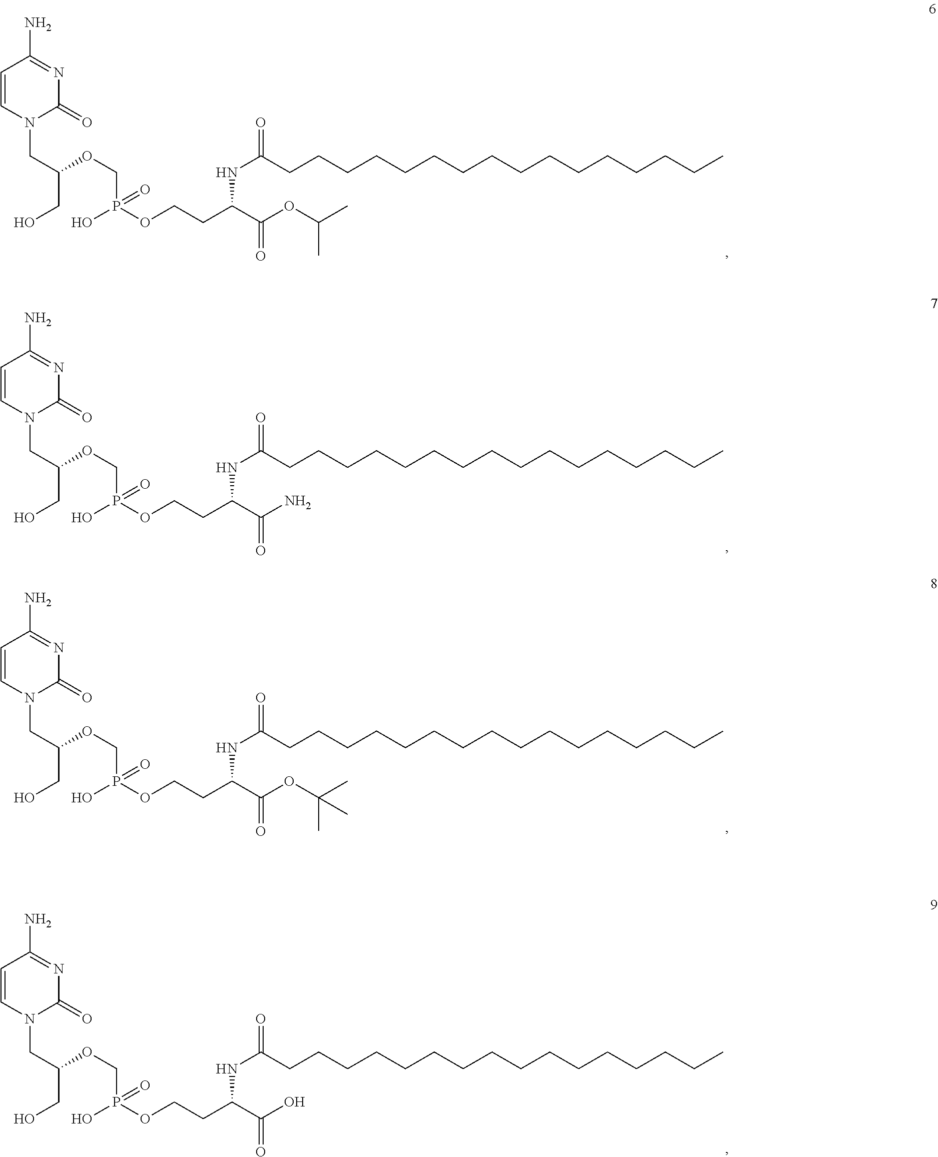 [SMILES: CCCCCCCCCCCCCCCCC(=O)N[C@@H](CCOP(=O)(O)CO[C@H](CO)Cn1ccc(N)nc1=O)C(=O)OC(C)C ; CCCCCCCCCCCCCCCCC(=O)N[C@@H](CCOP(=O)(O)CO[C@H](CO)Cn1ccc(N)nc1=O)C(N)=O ; CCCCCCCCCCCCCCCCC(=O)N[C@@H](CCOP(=O)(O)CO[C@H](CO)Cn1ccc(N)nc1=O)C(=O)OC(C)(C)C ; CCCCCCCCCCCCCCCCC(=O)N[C@@H](CCOP(=O)(O)CO[C@H](CO)Cn1ccc(N)nc1=O)C(=O)O] 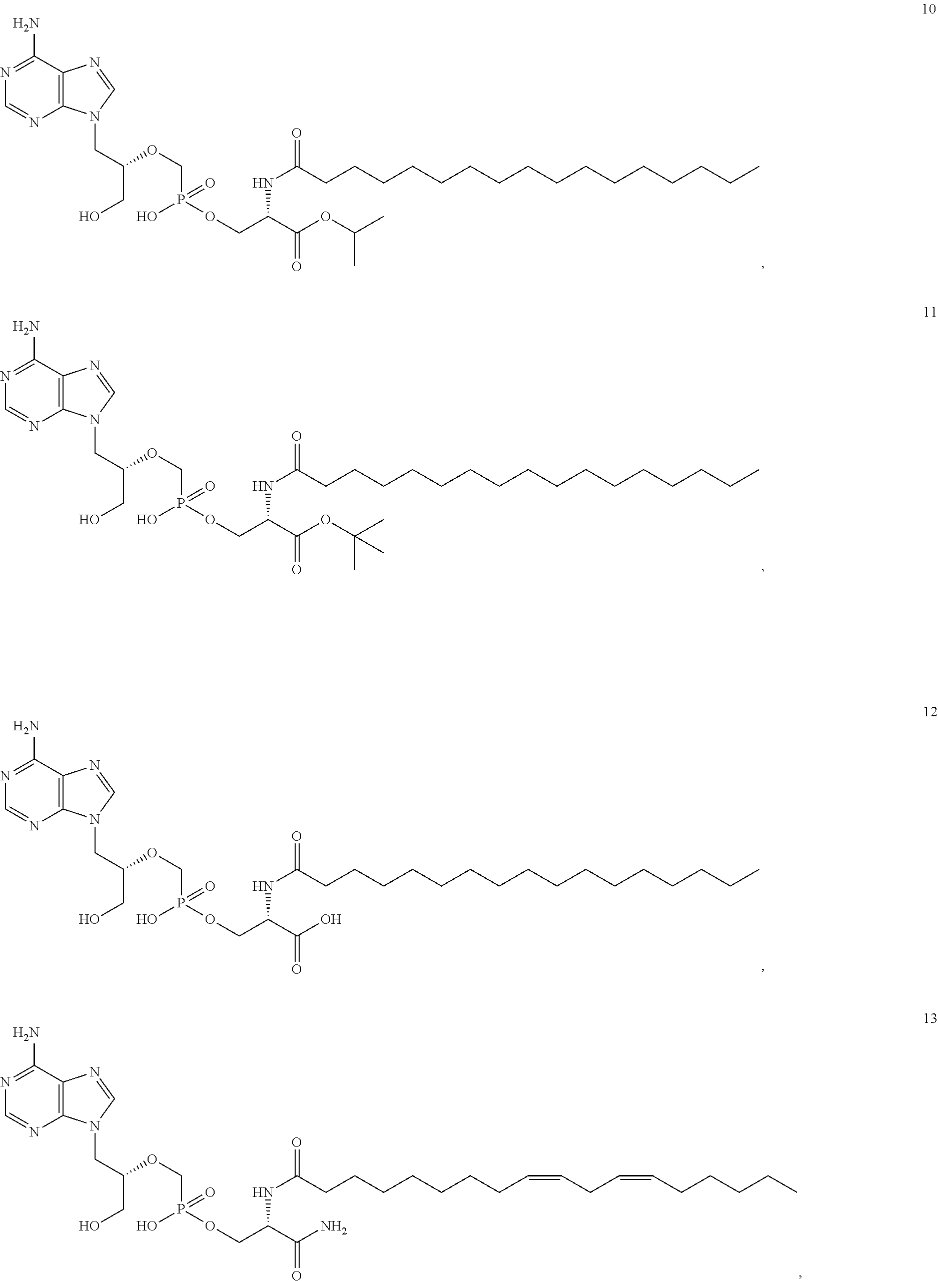 [SMILES: CCCCCCCCCCCCCCCCC(=O)N[C@@H](COP(=O)(O)CO[C@H](CO)Cn1cnc2c(N)ncnc21)C(=O)OC(C)C ; CCCCCCCCCCCCCCCCC(=O)N[C@@H](COP(=O)(O)CO[C@H](CO)Cn1cnc2c(N)ncnc21)C(=O)OC(C)(C)C ; CCCCCCCCCCCCCCCCC(=O)N[C@@H](COP(=O)(O)CO[C@H](CO)Cn1cnc2c(N)ncnc21)C(=O)O ; CCCCC/C=C\C/C=C\CCCCCCCC(=O)N[C@@H](COP(=O)(O)CO[C@H](CO)Cn1cnc2c(N)ncnc21)C(N)=O] 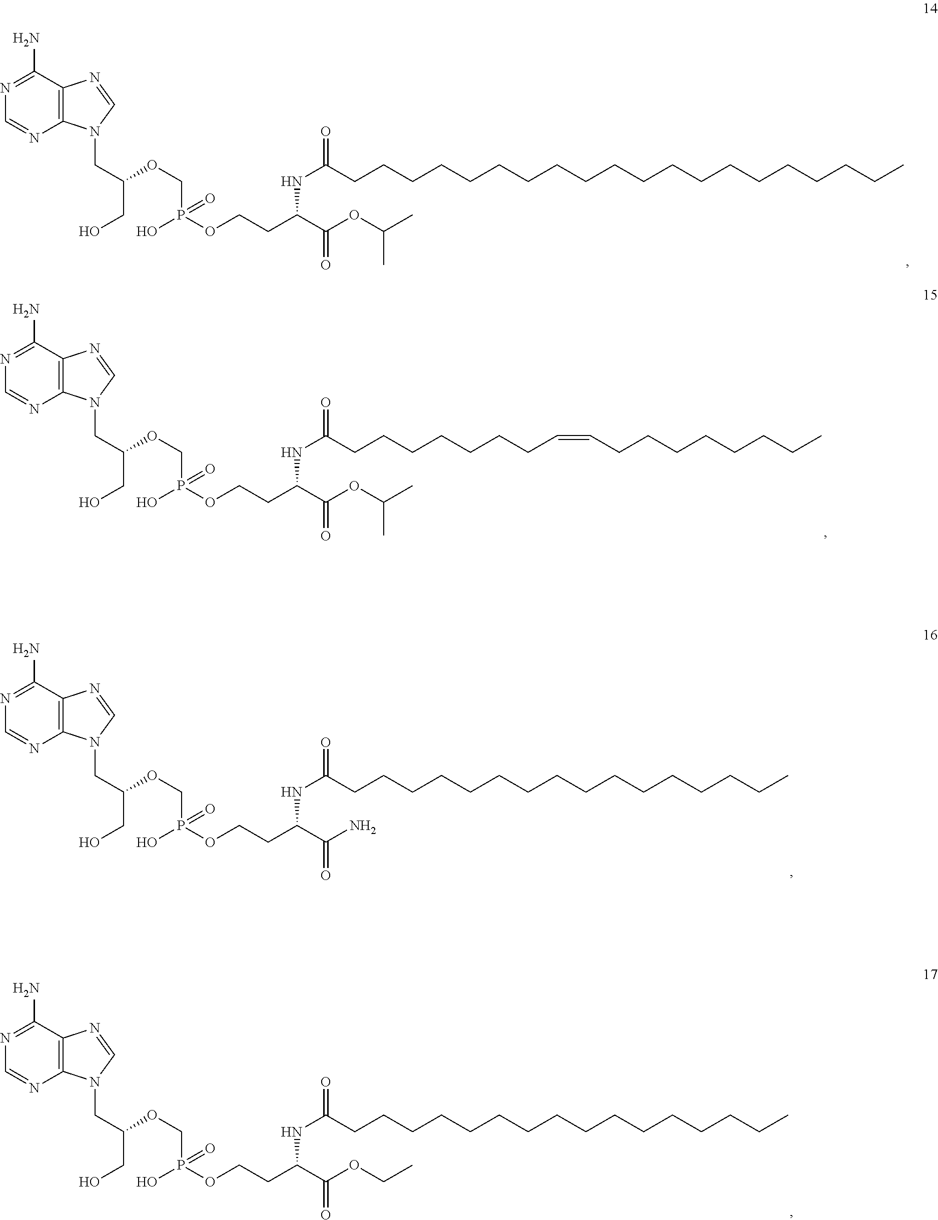 [SMILES: CCCCCCCCCCCCCCCCCCCCC(=O)N[C@@H](CCOP(=O)(O)CO[C@H](CO)Cn1cnc2c(N)ncnc21)C(=O)OC(C)C ; CCCCCCCC/C=C\CCCCCCCC(=O)N[C@@H](CCOP(=O)(O)CO[C@H](CO)Cn1cnc2c(N)ncnc21)C(=O)OC(C)C ; CCCCCCCCCCCCCCCCC(=O)N[C@@H](CCOP(=O)(O)CO[C@H](CO)Cn1cnc2c(N)ncnc21)C(N)=O ; CCCCCCCCCCCCCCCCC(=O)N[C@@H](CCOP(=O)(O)CO[C@H](CO)Cn1cnc2c(N)ncnc21)C(=O)OCC]
or a pharmaceutically acceptable salt thereof.

22. The prodrug of claim 1, wherein the amino acid residue linking D_{M} to X has the D-configuration.

23. The prodrug of claim 21, wherein X is CH₂CH₂ derived from D-homoserine and D_{M} comprises(S)-HPMPA.

24. A method of treating an adenovirus infection in a subject in need thereof, comprising administering to the subject a therapeutically effective amount of a prodrug comprising a compound having formula (1): 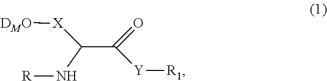
or a pharmaceutically acceptable salt thereof,
wherein:
D_{M} is a drug moiety;
X is selected from the group consisting of C₁-C₄ alkylene, C₆-C₁₀ arylene, and C₇-C₁₂ alkylarylene;
Y is O or NH;
R is H, C₁-C₆ alkyl, or C(O)R₂;
R₁ is H, C₁-C₂₅ alkyl, C₂-C₂₅ alkenyl, C₂-C₂₅ alkoxyalkyl, or C₂-C₂₅ alkylthioalkyl; and
R₂ is lipid-like.

25. The method of claim 24, wherein the prodrug is administered subcutaneously.
